(19) United States
(12) Patent Application Publication
(10) Pub. No.: US 2003/0232383 A1
(43) Pub. Date: Dec. 18, 2003

(54) Novel reagentless sensing system for measuring carbohydrates based on the galactose/glucose binding protein

(76) Inventors: Sylvia Daunert, Lexington, KY (US); Leonidas G. Bachas, Lexington, KY (US); Lyndon L.E. Salins, Lexington, KY (US)

Correspondence Address: BIRCH STEWART KOLASCH & BIRCH, PO BOX 747, FALLS CHURCH, VA, 22040-0747, US

(21) Appl. No.: 10/138,503

(22) Filed: May 6, 2002

Related U.S. Application Data
(60) Provisional application No. 60/330,905, filed on Nov. 2, 2001.

(51) Int. Cl. G01N 33/53; C07K 14/415; C07K 14/705

(52) U.S. Cl. 435/7.1; 530/395

(57) ABSTRACT

Galactose/glucose binding protein (GBP) is synthesized by Escherichia coli (E. coli) in a precursor form in the cytoplasm and exported into the periplasmic space upon cleavage of the 23 amino acid leader sequence. GBP binds galactose and glucose in a highly specific manner. The ligand induces a binge motion in GBP and the resultant protein conformational change constitutes the basis of the sensing system. Biosensors based upon GBP have been developed. These biosensors use various analytical signals, including option (i.e., fluoresecence) and electrochemical. The analytical methods were used to determine the amount of glucose present.

### CROSS REFERENCE TO RELATED APPLICATIONS

This application claims the benefit of U.S. Provisional Application No. 60/330,905 filed Nov. 2, 2001.

### GOVERNMENT INTEREST IN THE INVENTION

This invention was made with Government support under Grant No. NCCW-60 awarded by the National Aeronautics and Space Administration. The Government has certain rights in this invention.

### BACKGROUND OF THE INVENTION

Biosensors are chemical devices that are capable of detecting a particular analyte. In general, a molecule of biological origin (e.g., antibody, enzyme, or protein) serves as the biorecognition element that selectively binds the analyte producing an analytical signal (thermal, mass, electrochemical, or optical) that is proportional to the analyte concentration. Selective binding is the key to the biosensor concept of chemical analysis. It is important that the biorecognition element not only provides selectivity, but also a reasonably rapid release of the analyte to ensure reversibility of the sensor response. (Thompson et al., Anal. Chem. 1991, 63, 393A-405A).

Some of the current difficulties associated with glucose sensors include manufacturing reliability and consistency, short life spans, and cost. The number of biosensors with demonstrated capabilities for in vivo sensing is also limited (Wilson et al. Chem. Rev. 2000). Most sensors in the market today utilize glucose oxidase because the enzyme is stable and the cost of the enzyme is low. A major disadvantage of the glucose oxidase system is that a number of in vivo endogenous species of the enzyme are electroactive at the applied potential required for peroxide formation (Wilson et al. Chem. Rev. 2000). The production of H₂O₂, as a result of glucose oxidase activity, leads to the eventual disintegration of these sensors. Thus, there is a need for other reversible and cost-effective sensors for glucose that can detect the analyte in micromolar or lower concentrations. Current sensors have detection limits in the millimolar range because that is the physiological level of glucose in the blood. The present invention strives to provide an approach to glucose sensing that outperforms glucose oxidase with a reagentless sensing system that uses the selectivity present in nature in the form of the galactose/glucose binding protein (GBP).

Galactose and glucose uptake in E. coli is mediated by a periplasmic binding protein—galactose/glucose binding protein (GBP) (Scholle et al., Mol. Gen. Genet. 1987, 208, 247-253). The synthesis of GBP, the product of the mglB gene, can be induced by isopropyl-p-D-thiogalactoside (IPTG). GBP is synthesized in the cytoplasm in a precursor form with a signal sequence consisting of 23 N-terminus amino acid residues (Scholle et al., Mol. Gen. Genet. 1987, 208, 247-253). The function of these residues, most of which are hydrophobic, is to anchor the polypeptide to the inner membrane and enable the transportation of the protein to the space between the cell wall and the outer membrane (periplasm). During this process the signal peptide is cleaved off.

Mature GBP consists of 309 amino acids with a molecular weight of 33,310 Da (Mahoney et al., J. Biol. Chem. 1981, 256, 4350-4356). GBP is known to be involved in both active transport and bacterial chemotaxis, a process by which bacteria upon sensing a concentration gradient of a chemical substance move either towards or away from the substance. This mechanism of chemotaxis involves interaction of an exposed site located in one of the GBP domains with the transmembrane signal transducer protein, trg, which is responsible for triggering chemotaxis (Vyas et al., Science 1988, 242, 1290-1295).

As seen in FIG. 1, GBP is ellipsoidal in shape with two different but similarly folded domains connected by three different peptide segments that serve as a flexible hinge. Each domain has a core of six 13-sheet strands flanked by two or three helices on both sides (Vyas et al., J. Biol. Chem. 1991, 266, 5226-5237). In the absence of substrate, the two domains remain far apart with the cleft accessible to solvent. In the presence of the ligand, the two globular domains close with the three segments that connect the two domains acting as a hinge. In this bound-form, the domains are close to each other engulfing and burying the ligand. Thus, the sugar, D-glucose or D-galactose, is bound and completely engulfed in the deep cleft between the two domains. The exclusion of solvent molecules from the binding pocket enables efficient hydrogen bonding interactions between the substrate and the residues in the active site.

GBP binds to D-glucose and D-galactose with dissociation constants, K_{d}, of 0.2 mM and 0.4 mM, respectively (Miller et al., J. Biol. Chem. 1983, 258, 13665-13672). The aspartic acid residue at position 14 forms hydrogen bonds with the hydroxyl group on carbon 4 of the sugar when it is either in the equatorial position in D-glucose or in the axial position in D-galactose. This explains the fact that there is such negligible difference in the affinity of GBP for the two epimers. In the binding site, the sugar ligand is sandwiched between two aromatic residues (Phel6 and Trp 183) (Mahoney et al., J. Biol. Chem. 1981, 256, 4350-4356).

### BRIEF DESCRIPTION OF THE DRAWINGS

The present invention will become more fully understood from the detailed description given below, and the accompanying drawings which are given by way of illustration only, and thus are not limitative of the present invention, and wherein,

FIG. 1. Crystal structure of the ligand-bound galactose/glucose binding protein.

FIG. 2. Structure of the thiol-reactive fluorophores used in the labeling of the GBP mutants.

FIG. 3. DTPA isothiocyanate, a lanthanide-chelating molecule that can be attached to GBP and employed as a reporter in the sensing for glucose.

FIG. 4. Structure of anilinonaphthalenesulfonate (ANS).

FIG. 5. Example of the construction of a plasmid encoding for a CaM with a C-terminal cysteine residue.

FIG. 6. Purification of the modified CaM and introduction of a cysteine at the C-terminus of the protein.

FIG. 7: Construction of cytoplasmic and periplasmic GBP expression plasmids.

FIG. 8. Schematic for the expression of GBP.

FIG. 9. Purification of GBP via perfusion anion-exchange chromatography.

FIG. 10. SDS-PAGE (silver stained) analysis of GBP.

Lane 1 consists of the protein molecular weight markers.

Lane 2 indicates the crude periplasmic extract with GBP as the major band at ˜33 kDa.

Lane 3 shows GBP purified in a single chromatographic step.

FIG. 11: Reaction scheme for conjugation of 2-dimethylaminonaphthalene-5-sulfonyl chloride (D-22) to the lysine residues of GBP.

FIGS. 12A-12D: Steady-state emission spectra and quenching of terbium in wild-type GBP in the presence and absence of glucose (12A, 12C) or galactose (12B, 12D).

FIG. 13: Construction of mutant GBP expression plasmids.

FIG. 14: Crystal structure of GBP indicating the three positions selected for site-directed mutagenesis.

FIG. 15: Calibration plot for glucose and galactose using the MDCC-labeled GBP mutant at position 148.

FIG. 16: Calibration plot for glucose and galactose using the GBP mutant labeled with MDCC at position 152 (squares-glucose, diamonds-galactose).

FIG. 17: Steady state emission spectra of terbium coordinated to wild-type GBP in the absence and presence of glucose (squares-minus glucose, circles-plus glucose).

FIG. 18: Incubation time study for terbium-labeled GBP with glucose.

FIG. 19. Calibration curve for glucose obtained with terbium fluorescence of wild-type GBP.

### DETAILED DESCRIPTION OF THE INVENTION

The resultant conformational change that accompanies binding of a carbohydrate, typically galactose or glucose, by GBP forms the basis of the sensing system of the present invention. When GBP is labeled to provide an analytical signal (thermal, mass, electrochemical, or optical), a change in the signal is seen when the glucose ligand binds GBP and induces a change in the conformation of the protein. This change in signal can then be related to the concentration of carbohydrate in the sample.

In the present invention, two different strategies were employed to design sensitive sensing systems for the ligand. The first involved the site-specific introduction of a unique cysteine residue at positions in the protein that might experience changes in the local environment when GBP undergoes structural changes as a result of the binding event. Labeling these cysteine residues with sulfhydro-specific probes enabled the quantification of the conformational change, which can be related to the amount of ligand present in the solution. The second method made use of the wild-type protein, which has a unique calcium binding site. No alterations were made to the sequence of the protein. Rather the calcium ion normally bound to GBP is replaced with a lanthanide series ion. In one embodiment, the terbium ion, a fluorescent lanthanide series ion, was placed in the binding site and used as a reporter.

The unique calcium binding site in GBP is in the C-terminal domain at one end of the ellipsoidal protein molecule. The function of calcium is thought to confer stability to the protein structure. The Ca²⁺ is coordinated to seven protein oxygen atoms in a pentagonal bipyramid geometry. A nine-residue loop consisting of amino acids 134 to 142 surrounds the calcium binding site and provides five of the seven oxygen atoms that coordinate to the Ca²⁺. The remaining two oxygens are provided by the carboxylate group of Glu205 (Vyas et al., Nature 1987, 327, 635-638). It has been reported previously, in the case of other binding proteins, that replacement of Ca²⁺ by other metals, such as lanthanum (La), Yttrium (Y), or Cerium (Ce), renders proteins that are still active (Martin et al. Q. Rev. Biophys. 1979, 12, 181-209; Horrocks et al. Acc. Chem. Res. 1981, 14, 384-392; Bruno et al. Biochem. 1992, 31, 7016-7026; Selvin, P. R. Methods in Enzymol. 1995, 246, 300-334).

Replacement of the calcium with a lanthanide series ion, such as terbium (Tb) or europium (Eu), ensures a unique site for reporting conformational changes that accompany binding. In a preferred embodiment, measurement of a fluorescent signal for the ion can detect the protein conformational change associated with the binding of the ligand. The change in fluorescent signal that occurs when a fluorescently labeled protein binds to a ligand is a valuable tool for the detection of that particular ligand. Typically, either the fluorescent intensity or lifetime is measured. In another preferred embodiment, an electrochemical signal is measured.

As used herein, the term “carbohydrate” refers to a monosaccharide, disaccharide, oligosaccharide or polysaccharide. Preferably, a monosaccharide is detected. More preferably, a six-carbon sugar is detected. Still more preferably, glucose or galactose is detected by the method or sensors of the present invention.

Any reporter label may be used so long as it can be attached to a mutant GBP protein or compete with the calcium ion on the wild-type GBP protein, is assayable, and the signal label is related to the amount of the specific carbohydrate bound to GBP. The environmentally sensitive dansyl family of fluorescent probes is known to respond to changes in local environment and are non-fluorescent until reacted with amines. It is a preferred embodiment of the invention that a lanthanide series ion, such as La, Ce, Pr, Nd, Pm, Sm, Eu, Gd, Tb, Dy, Ho, Er, Tm, Yb or Lu be used as the label. More preferably, europium (Eu) or terbium (Tb) is used in the method of the invention. In this regard, any assay method may be used. Preferably, the signal may be detected by fluorescence, luminescence, chemiluminescence or electrochemical methods.

As used herein, “carbohydrate binding protein” refers to any protein that can bind to a specific carbohydrate in a calcium dependent manner, and any variants or analogs of such proteins. Examples of such proteins include lectins, the carbohydrate binding domains of lectins, carbohydrate receptors, and galactose/glucose binding protein. Of the lectins, C-type lectins are preferred. The carbohydrate recognition domain of C-type lectins consists of about 130 amino acids, and requires disulfide-linked cysteines and calcium ions in order to bind to a specific carbohydrate. In addition to lectins, there are several carbohydrate binding proteins and carbohydrate recognizing enzymes that bind carbohydrates. For example, cyanovirin-N binds to the HIV viral envelope glycoprotein gp120 thus inhibiting viral entry.

As used herein, “galactose/glucose binding protein (GBP)” refers to a protein that specifically binds to galactose or glucose, and includes variants and mutants thereof, so long as the binding protein binds specifically to glucose or galactose and allows signaling from a label in proportion to the amount of glucose or galactose present in a sample, wherein the intensity of the signal varies with the amount of glucose or galactose present in the sample. Preferably, the GBP used is a gene product of the bacterial mglB gene or a derivative thereof.

In the method of the invention, glucose or galactose can be detected in any liquid sample, such as blood, urine, culture media, environmental samples, interstitial fluid, bodily fluids or any other liquid sample. Further, the invention may be practiced as a kit for performing the method. Thus, the invention includes an article that comprises written instructions, or directs the user to written instructions, for how to practice the method of the invention or use the sensors of the invention.

As used herein, “reagentless” assay means that no additional substrate needs to be added to the reaction to monitor the amount of carbohydrate in the sample.

Bacterial periplasmic binding proteins are proteins that serve as initial receptors for active transport systems (Furlong, C. E., Escherichia coil and Salmonella typhimurium: Cellular and Molecular Biology, Neidhardt, F. C., ed.; American Society of Microbiology: Washington, D.C., 1987, pp. 768-796; Ames, G. F. L. Annu. Rev. Biochem. 1986, 55, 397-425). Ligands cross the outer membrane non-specifically and bind to periplasmic binding proteins with high affinity with a K_{d} in the μM range. When the ligand is bound to the protein, the ligand-protein complex interacts with a membrane-bound transport complex. This triggers the release of the ligand and its subsequent translocation into the cytoplasm accompanied by ATP hydrolysis (Williams et al., J. Biol. Chem 1989, 264, 7536-7545).

Ligand transport in Gram negative bacteria like E. coli and Salmonella is mediated by periplasmic binding proteins. There are over two dozen such proteins which serve as an uptake system for sugars, oxyanions, amino acids, and oligopeptides (Wilson et al., Bacterial Transport, Rosen, B. P., ed.; Marcel Dekker: New York, 1978, pp. 495-557; Furlong, C. E., Escherichia coil and Salmonella typhimurium: Cellular and Molecular Biology, Neidhardt, F. C., ed.; American Society of Microbiology: Washington, D.C., 1987, pp. 768-796). They consist of a single polypeptide chain with a tertiary structure in the form of two globular domains connected by three short peptide segments (Sack et al., J. Mol. Biol. 1989, 206, 171-191). The ligand binding site is located at the base of the cleft between the two domains. Binding of the ligand is accompanied by the closing of the cleft with the segments acting as a hinge.

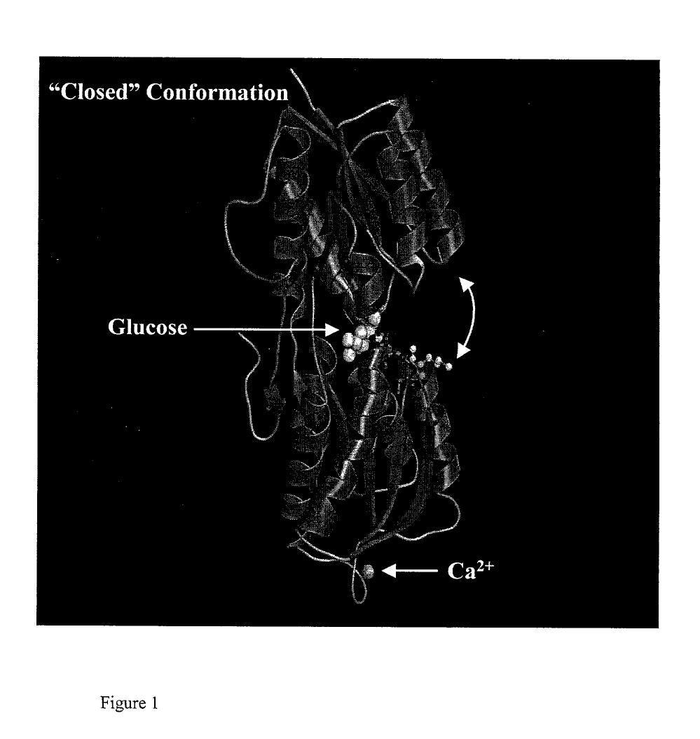

Galactose and glucose transport in E. coli is mediated by periplasmic GBP, which binds the ligand in a highly specific manner. In the absence of the ligand, the two domains remain far apart with the cleft accessible to solvent. The ligand binding site is deep within this cleft. In the presence of the substrate, the two globular domains close with the three segments that connect the two domains acting as a hinge (FIG. 1). In this bound-form, the domains are close to each other engulfing and burying the ligand. The exclusion of solvent molecules from the binding pocket enables efficient hydrogen-bonding interactions between the substrate and the residues in the active site. Binding specificity and affinity are conferred primarily by polar planar side-chain residues that form intricate networks of cooperative and bidentate hydrogen bonds with the sugar substrates, and secondarily by aromatic residues that sandwich the pyranose ring (Vyas et al. Science 1988, 242, 1290-1295).

Steady-state fluorescence studies by the inventors indicated that the hinge motion and binding properties of GBP could be utilized with reporter molecules to develop reagentless fluorescence-based biosensing systems for glucose. Thus, the resultant conformational change that accompanies ligand binding in GBP forms the basis of the present invention. Upon ligand binding to GBP, a change in the conformation of the protein is induced. By attaching a reporter that is sensitive to the local molecular environment to the correct site of GBP, it is possible to perform measurements of carbohydrate by monitoring the change in the emitted signal of the reporter. This change can then be related to the concentration of carbohydrate, e.g., glucose or galactose in the sample. Using this system, the present inventors developed biosensing systems that are sensitive to submicromolar concentrations of glucose. Two reagentless sensing schemes for glucose were designed and developed using wild-type as well as the mutant forms of GBP.

When using a fluorophore as the reporter, in order to obtain maximal signal vs. background fluorescence, it is important that the fluorophore be attached to a site and at a position where maximum conformational change occurs. Through the use of modem protein engineering technology, the inventors chose the position to modify a single amino acid residue for the site-selective covalent attachment of environment-sensitive fluorophores. The edge of the cleft is an ideal position since in the process of the two domains closing, these sites experience a change in the local environment. Since wild-type E. coli GBP has no cysteine moiety, incorporation of a unique cysteine on the protein molecule permits selection of a specific site and allows for the optimization of the induced fluorescence change of the fluorescently-labeled protein in the presence of galactose and glucose.

The environmentally sensitive dansyl family of fluorescent probes is known to respond to changes in local environment and are non-fluorescent until reacted with amines. The initial fluorophore used in the present invention as a probe to measure galactose and glucose concentrations was 2-dimethylamino-naphthalene-5-sulfonyl chloride (D-22) (Haugland, R. P. Molecular Probes Handbook of Fluorescent Probes and Research Chemicals; Larison, K. D., ed.; Molecular Probes, Inc.: Eugene, Oreg., 1992; pp. 34-35), which is an isomer of dansyl chloride. Using this probe for conjugation results in multiple-site labeling since there are 22 lysine residues in GBP.

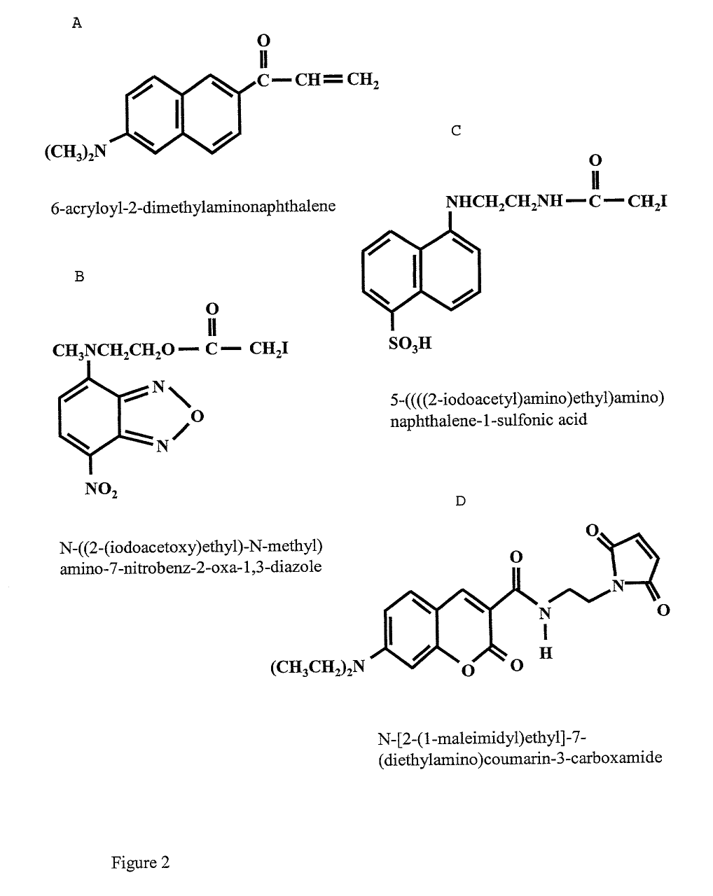

In another embodiment of the present invention, site-directed mutagenesis to incorporate a single cysteine in GBP was performed using overlap extension PCR. Three sites, Gly148, His152, and Met182, were chosen for site-directed mutagenesis based on the crystal structure of the protein to produce three different GBP mutants. The cysteine residue of each protein was labeled with fluorophores (FIG. 2) such as 6-acryloyl-2-dimethylaminonaphthalene (acrylodan), 5-((((2-iodoacetyl)amino)ethyl)amino)naphthalene-1-sulfonic acid (1,5-IAEDANS), N-[2-(1-maleimidyl)ethyl]-7-(diethylamino)coumarin-3-carboxamide (MDCC), and N-((2-iodoacetoxy)ethyl)-N-methyl)amino-7-nitrobenz-2-oxa-1,3-diazole (IANBD ester). MDCC and acrylodan are fluorescent probes that react with the thiol groups of cysteine residues forming a thioether bond. The response of the system, upon ligand binding, was monitored by following the changes in the fluorescence intensity of the probes. Calibration plots were then constructed by relating the changes in signal with the amount of ligand present.

Covalent binding of a molecule through the sulfhydryl group of a unique cysteine is not the only means by which a fluorescent probe can be attached to a protein. In the case of GBP, intrinsically fluorescent lanthanides may be complexed by the calcium-binding site of the wild-type protein and act themselves as environmentally sensitive probes. Studies by Vyas et al. (Vyas et al., J. Biol Chem 1989, 264, 20817-20821) have indicated that terbium has nearly equal binding affinity as calcium for the calcium binding site when compared with several other divalent metal ions. The dissociation constant for calcium is 2 μM and the dissociation rate for terbium was determined to be 1×10⁻³s⁻¹. Proteins that bind calcium have been found to bind terbium stoichiometrically and specifically without inducing significant structural changes and to enhance the lanthanide's fluorescence due to Forster dipole-dipole energy transfer from aromatic residues near the binding site. The net charge of the calcium bind site, −3, is able to stabilize the added charge of terbium. Therefore, size of the ion is the determining factor for fitting into the binding pocket. Unlike other calcium binding loops, such as that of calmodulin, the one in GBP does not cause the protein to undergo a significant conformational change upon cation binding. Another benefit of using terbium is its sensitivity to changes in the environment around the calcium binding site. The sugar-binding site can be found approximately 30 Å from the Ca²⁺ binding site and the tryptophan residue nearest the bound Ca²⁺. The sugar binding site and calcium binding site are found to be functionally independent. The present invention allows for the use of the wild-type protein, incorporating a reporter moiety into the native calcium binding site.

Thus, in yet another embodiment of the present invention, Ca²⁺ in the unique calcium-binding site of GBP was replaced with a lanthanide series metal, such as La, Y, Ce or Tb. These metals, which are naturally fluorescent, allow the reporting of conformational changes that accompany binding by monitoring the changes in the fluorescent properties of the protein molecule. In one example, the fluorescence intensity of the lanthanide series metal upon glucose binding was measured. In that example, the present inventors replaced the Ca²⁺ in GBP with a terbium or europium ion, which allowed reporting of the conformational changes that accompany binding by monitoring change in intensity of the emitted signal. A calibration plot was then constructed by relating the changes in signal with the known amount of glucose or galactose present in a sample. The amount of glucose or galactose in an unknown sample can then be determined by reference to the calibration plot.

Electronic implementation of the calibration and subsequent quantitation of an unknown sample can be implemented as known in the art.

This is the first time that the inherent fluorescence signal of a lanthanide is being employed to probe for ligand-induced conformational changes of binding proteins. Furthermore, as the lifetime of the fluorescence of lanthanides is also modulated by the environment of the ion, it is also possible to relate the change in fluorescence lifetime to the amount of glucose present in the sample. Since lifetime measurements are independent of the concentration of the reporter moiety (Lakowicz, J. R. In Principles in Fluorescence Spectroscopy; Plenum Press: New York, 1999; chap. 3, pp. 87-88), these sensors could potentially have extended service lives compared to currently employed glucose sensors. This, in turn, should render a new generation of in vivo sensors that can be used for the continuous monitoring of glucose.

While the selectivity of a sensing system is a function of the dissociation constant of the protein-ligand complex, the choice of analytical method is one the factors that influence sensitivity. In sensing systems that use isolated proteins as the sensing element, fluorophores are normally employed and the changes in the fluorescent intensity can be related to the concentration in the analyte.

Fluorescent techniques for determining fluorescent intensity are well known in the art. (Skoog, D. A., et al., Principles of Instrumental Analysis, 5th Edition, Saunders College Publishing, Philadelphia, 1998.) Further, the instruments used for fluorescence detection include, but are not limited to: typical benchtop fluorometers, which are available from vendors such as, Perkin Elmer (Shelton, Conn.) and Spex Jobin Yvon Inc (Edison, N.J.); fluorescence multi-well plate readers, such as the Cytofluor Systems that is available from Applied Biosystems (Foster City, Calif.); fiber optic fluorometers, which are commercially available from a number of sources, including Oriel Instruments (Stratford, Conn.); fluorescence microscopes, which are commercially available from vendors such as Nikon (Melville, N.Y.), Olympus, and Zeiss; and microchips/microfluidics systems coupled with fluorescence detection (e.g., the systems from Tecan-Boston (Medford, Mass.), Aclara Biosciences (Mountain View, Calif.)). In addition to fluorescence, it is also possible to employ other detection methods.

pH-Sensitive Electrochemical Probe for the Detection of Glucose Employing GBP

Electrochemical compounds can be used to label proteins and subsequently report changes in the target protein microenvironment. It is well established that solutions of electrochemical compounds demonstrate different formal potentials depending on solvent parameters. Among these parameters, hydrogen bonding, electrostatic interactions, hydrophobicity/hydrophilicity, solvent donor number (DN), etc. can affect the formal potential. In the case of charged compounds, the pH of the solvent is important as well. Shifts in the formal potential can then be related to the analyte-induced conformational change of the protein.

Although an electrochemical label that is sensitive to hydrophobicity/hydrophilicity or DN may be appropriate for monitoring protein conformational changes, it is certainly feasible and possibly advantageous to employ a label that is pH-sensitive instead. Indeed, it was shown previously with a maltose-binding protein labeled with a DN-sensitive compound that a change of <14 mV resulted upon binding of glucose (Trammell, S. A., Goldston, H. M., Tran, J. P. T., Tender, L. M., Bioconjugate Chem., 2001, 12: 643-647). This is a relatively small shift in formal potential, which makes practical application of this approach cumbersome. Another disadvantage of the electrochemical label of Trammell et al. is its bulkiness. This prohibits the label to reside within hydrophobic clefts of the protein, which are among the environments that undergo significant conformational change upon binding of a ligand to its corresponding binding protein.

The ideal pH-sensitive electrochemical probe to be used for monitoring protein conformational changes will have an ionizable group, be of geometry that can allow it to reside within hydrophobic clefts, have a group that will allow coupling to a residue on the surface of the binding protein, and have a formal potential that shifts in response to conformational changes in the targeted protein. Several classes of compounds fit this role, including ferrocenyl amines/carboxylates (Gleria, K. D., Hill, H. A. O., Wong, L. L., FEBS Lett., 1996, 390: 142-144), o-quinones, hydroquinones, and organometallic complexes (Trammell, S. A., Goldston, H. M., Tran, J. P. T., Tender, L. M., Bioconjugate Chem., 2001, 12: 643-647). Additionally, derivatives of pyrroloquinoline quinone (PQQ), topaquinone and other naturally occurring electrochemical cofactors can also be used. Through numerous amino acids, PQQ (shown in its reduced form) can be coupled to a protein (e.g., through amine groups on lysines).

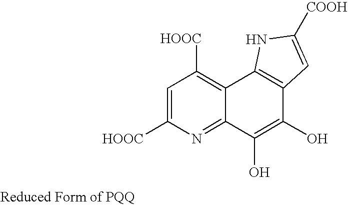

Labels can be used to direct coupling of an electrochemically active component at targeted sites on the protein. Because of the presence of charged groups on the labels (e.g., amines, quinoline hydroxyls, carboxylates), a conformational change in the protein will alter electrostatic interactions between the label and the protein and/or modify the local pH around the label. Both these factors will significantly change the formal potential registered by the label and give an electrochemical signal as a result of the conformational change. Thus, any analyte-induced change in the three-dimensional structure of the protein can be monitored leading to a highly sensitive and selective electrochemical biosensor.

These labels below are versatile and can be synthesized to direct coupling to a class of amino acid residues. In the two examples provided, maleimido moieties are introduced to facilitate attachment of the label to free thiols (from cysteine residues) on the protein. However, these are only used here as examples of general classes of compounds and different spacers, electroactive groups, charged or hydrogen-bonding residues, and functionalities for attachment to the protein can be employed as known in the art. The signal is electrochemical in nature. It is generated by the label and can be monitored by one of several voltammetric/amperometric methods (Bard et al., Electrochemical Methods: Fundamentals and Applications, Wiley, New York, 2000; Wang, Analytical Electrochemistry; Wiley, New York, 2000; Kissinger et al., Laboratory Techniques in Electroanalytical Chemistry, Marcel Dekker, 1996). Further, for electrochemical detection, there are many commercially available systems, such as the electrochemistry stations from Bioanalytical Systems, Inc. (West Lafayette, Ind.). The same vendor also sells portable miniaturized electrochemistry stations.

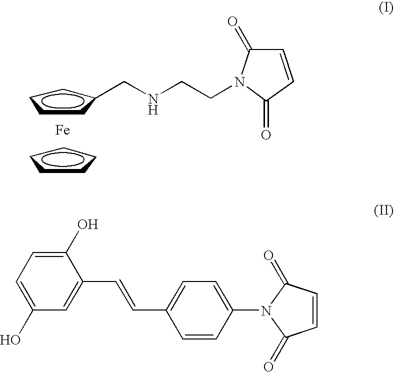

Structure (I): Ferrocyanylmethyl-ethylaminemaleamide

Structure (II): trans-2′,5′-dihydroxystilbene-4-maleamide

To make ferrocyanylmethyl-ethylaminemaleamide, commercially available ferrocene carbaldehyde and excess ethane-1,2-diamine are refluxed in ethanol to form the corresponding imine. Reduction of the imine with LiAlH₄ (M. J. L. Tendero, A. Benito, R. Martinez-Manez, J. Soto, J. Paya, A. J. Edwards, P. R. Raithby, Chem. Soc., Dalton Trans., 1996, 343) in freshly distilled tetrahydrofuran produces 1,4-diazapentyl ferrocene. The latter will be reacted with maleic anhydride to form the corresponding maleimide shown above.

The synthesis of trans-2,5-dimethoxy-4′-aminostilbene has been described previously (X. Yang, S. B Hall, A. K. Burrell, D L. Officer, Chem. Commun., 2001, 2628-2629). This will be reacted with maleic anhydride to formed the corresponding trans-2,5-dimethoxy-4′-maleimidostilbene. The electrochemical label will be formed by conversion of the methoxy groups to hydroxyls through treatment with BBr₃ in methylene chloride.

Glucose Biosensor Employing Lanthanide Complexing Agents

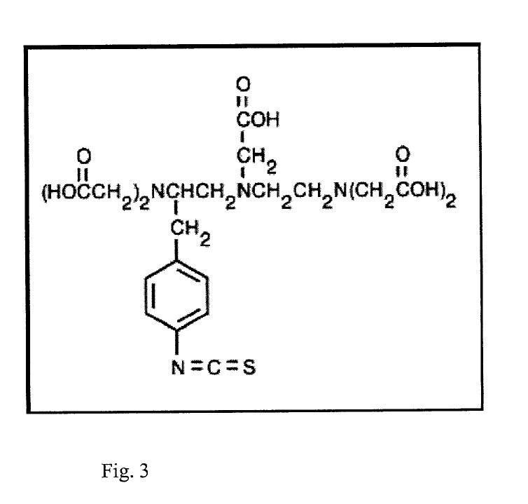

Lanthanide complexing agents may also be used as reporter molecules in the detection of glucose with GBP. The rationale for employing lanthanide complexing agents as reporter molecules is similar to that explained for the use for a lanthanide ion as the reporter. In this case, in order to better attach the lanthanide ion to the protein, while retaining its time-resolved capabilities, and prevent leaching, a chelating moiety can be used to attach the lanthanide reporter to the protein with a higher affinity than the protein's native calcium site can offer. Various appropriate complexing agents and methods for complexing agents to proteins are known in the art. (Dickson, E. F., A. Pollak, and E. P. Diamandis, Pharmacol. Ther., 1995. 66, 207-235; Dickson, E. F., A. Pollak, and E. P. Diamandis, J. Photochem. Photobiol. B, 1995. 27, 3-19; Hemmila, I., Scand. J. Clin. Invest., 1998, 48, 389-399; Seveus, L., et al., Cytometry, 1992. 13, 329-338.; Niemi, P., et al., Invest. Radiol., 1991. 26, 820-824; Hemmila, I. A. and H. J. Mikola, Acta. Radiol. Suppl., 1990. 374, 53-55; Markela, E., T. H. Stahlberg, and I. Hemmila, J. Immunol. Methods, 1993. 161, 1-6; Suonpaa, M., et al., J. Immunol. Methods, 1992. 149, 247-253). Thus, after the initial complexing of the ion with the complexing agent, there is no need for the labeled protein to always be considered in a solution containing the lanthanide series ion solution to always be in contact with the protein to avoid leaching. The principle of emission of a signal will be the same as described when using the lanthanide series ion incorporated directed into the calcium-binding site. There are a series of complexing agents that are bifunctional and could be used in our approach. An example is provided in the diethylenetriaminepentaacetic acid (DTPA) isothiocyanate structure, where the carboxylate moieties complex the lanthanide ion and another functionality, namely the isothiocyanate, that can be used to attach the complexing agent to an amino acid residue on the protein. The amino acid residue for attachment (a lysine, a cysteine, etc.) depends on the type of functional group the complexing agent has for attachment to the protein. In FIG. 3, the structure shown has an isothiocyanate molecule that can be employed in the labeling of lysine residues on the protein. This is well-established conjugation chemistry and DTPA isothiocyanate has been employed as a bifunctional chelator when attached to a chromophore as well as the lanthanide of interest (Vereb et al. Biophys. J. 1998, 74, 2210-2222; Hemmila et al. Acta Radiol. Suppl. 1990, 374, 53-55; Markela et al. J. Immunol. Methods 1993, 161, 1-6; Heyduk and Heyduk Anal. Biochem. 1997, 248, 216-227). It has been demonstrated that that the iosthiocyanate derivative of DTPA yields conjugates that retain all five carboxylate groups resulting in more stable metal complexation to the lanthanide series ion. Other molecules that can be used for these purposes include complexing agents such as DTPA-cs124 (Deal et al., J. Med Chem. 1996, 39, 3096-3106) and 6-substituted 2,4-dichloro-1,3,5-triazines (Karsilayan et al. Bioconjugate Chemistry 1997 8, 71-75 and references therein). Alternative attachment methods include those commercially available from Perkin Elmer Life Sciences where an oligonucleotide is essentially used as a spacer or anchor between the protein and the lanthanide complexing agent (see Perkin Elmer Life Sciences webpage).

Organic Compounds that Bind to Hydrophobic Pockets

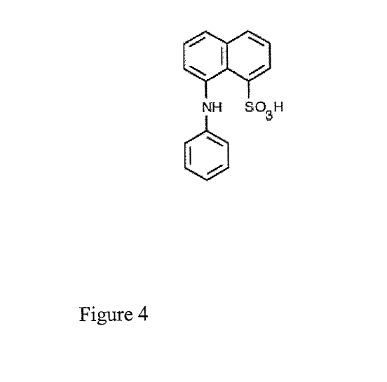

The inventors identified a hydrophobic region in the molecule of GBP by studying the X-ray crystal structure of GBP bound to glucose. This hydrophobic region is near the C-terminal region of GBP and close to the calcium-binding site. A new approach to sensing utilizing this hydrophobic region of GBP involves the exposure of GBP to an organic compound (preferably planar in structure) capable of generating a signal. When the organic compound is exposed to GBP, it binds to the hydrophobic region of the protein though various non-covalent interactions, such as π-π interactions. Glucose binding to GBP causes an allosteric effect on the calcium-binding site, which is in proximity to the hydrophobic region containing the signal-generating organic molecule. Because of this proximity, the binding of glucose to GBP also affects the properties of the signal-generating organic molecule in a manner that is proportional to the amount of glucose present. The organic molecule generates a change in fluorescence intensity. In this approach, there is no need for chemical or genetic modification of GBP. Native GBP without modification is used. Examples of molecules that could be employed include anilinonaphthalenesulfonate (ANS), which has an excitation λₘₐₓ of 360 nm and an emission λₘₐₓ of 470 nm. (see FIG. 4). In the case where the calcium molecule is replaced with a lanthanide series ion, the emission from the lanthanide series ion could excite the organic compound. Then, the detection is observed at the emission wavelength of the organic compound.

Fusion of a pH Sensitive Protein (for Example, EGFP) with GBP to Create a Chimeric Protein where a Change in Signal is Observed Due to Change in pH Microenvironment upon Binding of the GBP Portion of the Chimera to Glucose.

The preparation of a GBP sensing molecule that has an integrated signal-generating molecule in its structure can be accomplished by fusing the gene of native GBP either from the C- or the N-terminus with that of a protein/peptide molecule, such as Enhanced Green Fluorescence Protein that is very sensitive to changes in pH. The GBP can be directly fused to the N- or C-terminus of the signal-generating peptide/protein or an amino acid linker of variable length and composition can be placed in between both proteins. The signal-generating peptide/protein is of the kind that when the pH of its microenvironment changes causes a measurable change of another property of the molecule results. Alternatively, a shift in the pKₐ of residues in the vicinity of the chromophore can result in a change in signal. The latter can be achieved through conformational changes of the protein that alter local interactions among amino acids, i.e, the protein EGFP changes its fluorescence emission when the pH of its microenvironment changes (Deo, et al., S., Anal. Biochem., 2001, 289, 52-59). This is an alternative means of creating a GBP protein that is labeled with a signal-generating molecule that will cause the emission of a signal upon glucose binding. The advantages include the creation of a homogeneous population of the glucose-sensing GBP with the integrated signal-generating molecule. The possibility of creating a homogeneous population allows for an increased reproducibility in the lot-to-lot preparation of the biosensor protein reagent, which ultimately leads to an increased reproducibility in the biosensor response. In addition, the produced protein because it is prepared by genetic means, it can also be produced and subsequently used to sense glucose “in vivo” without the need for any external reagents for signal generation. Another method of preparation of a protein that contains GBP and the signal-generating peptide/protein, such as EGFP, is by attaching the EGFP molecule to another amino acid residue other than the N- or C-terminus of GBP. In this case the method does not involve genetic fusion, but rather a chemical coupling of GBP with EGFP. This can be accomplished by different methods of attachment including those explained earlier that involve cysteine-mediated coupling.

Attachment of GBP to Surfaces through Cysteine Groups on the GBP Molecule

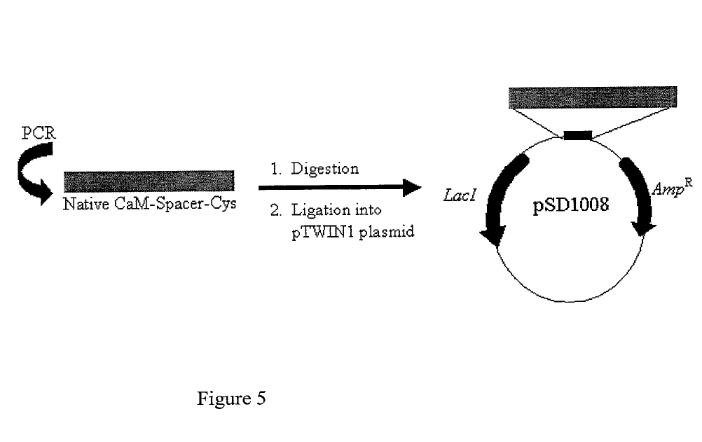

The GBP protein can be site-specifically immobilized on a solid surface. For that, a unique cysteine can be introduced at the C- or N-terminus of the native GBP, which does not contain any cysteine molecule, using molecular biology techniques. In addition, the cysteine molecule could be introduced at other sites on the GBP molecule where the conformational change that the protein undergoes upon binding to glucose will not be evident. The polymerase chain reaction (PCR) can be employed to construct the gene that codes for an otherwise native GBP with a unique cysteine at the C-terminus. An amino acid spacer that can have from different lengths (e.g., five amino acid spacer such as Ser-Gly-Gly-Gly-Ser), etc.) can be also introduced between GBP and the cysteine residue to allow flexibility for GBP to bind to glucose when immobilized on a surface. The DNA obtained from the PCR reaction can then be ligated into an expression vector, such as the pTWIN1 vector to yield a plasmid that will encode for the desired GBP with a terminal cysteine. The present inventors have demonstrated the feasibility of using this approach for the preparation of such an expression vector for the modified protein by employing the calcium-binding protein calmodulin (for an example of such an expression vector see FIG. 5).

The plasmid pSD137 can be transformed into E. coli cells and the protein expressed by inducing the cells with, for example, isopropyl-β-thio-galactopyranoside (IPTG). The protein can be purified by employing ion-exchange and/or affinity chromatography.

This N-or C-terminal modified protein with the cysteine residue can be used directly for immobilization on a surface when the biosensor is based on the strategy that involves the incorporation of a lanthanide molecule to replace calcium in the allosteric site of GBP. However, in the case where the biosensor is based on a signal-generating molecule, a specific site for attachment of this molecule can be incorporated in addition to the site for anchoring of the GBP to a surface.

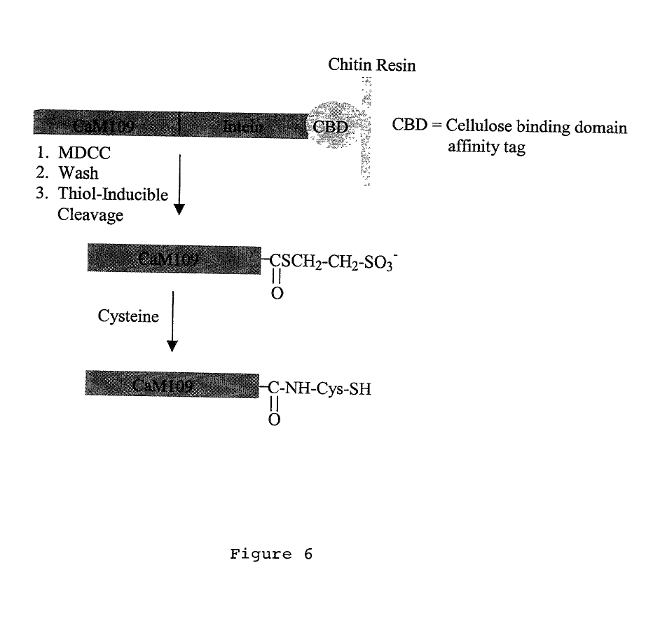

In the case where it is desired to introduce another cysteine residue for attachment of a signal-generating molecule, a cysteine residue can be introduced in a particular location on the protein by using the PCR-based method described above. Once this modified protein is obtained, in order to immobilize the modified GBP site-specifically to a surface, a cysteine can be introduced at its N- or C-terminus using commercially available molecular biology reagents, such as the IMPACT-TWIN (Intein Mediated Purification with an Affinity Chitin-binding Tag-Two intein) system from New England Biolabs (Beverly, Mass.). The IMPACT-TWIN system utilizes the inducible self-cleavage activity of the protein splicing elements termed inteins to separate the target protein from the affinity tag. Upon cleavage it yields a reactive thioester linkage at the C-terminus of the target protein. We utilize this property to introduce a cysteine molecule at the C-terminus of the modified GBP that allows one to perform site-specific immobilization. For that, the gene for the modified GBP is ligated into an expression vector, such as the pTWIN1 vector. A signal-generating molecule can then be conjugated to the cysteine in the desired position before inducing the cleavage of the affinity tag. After the cleavage, the amino acid cysteine can be added which can react with the thioester linkage yielding a cysteine molecule at the C-terminus of the modified GBP molecule. The present inventors have demonstrated that this approach works by using this strategy to prepare a calmodulin protein modified in position 109 (where a signal generating molecule was attached) that also contains a cysteine molecule at the C-terminus for anchoring to a solid surface (see FIG. 6 for the example).

The next step involves the immobilization of the GBP (with and without the signal generating molecule) with a cysteine at the C-terminus on a solid surface. The nature of the surface can be diverse, e.g., silica, a hydrogel, a sol-gel, a polymer film, a membrane, self-assembled monolayers, a Lagmuir-Blodgett film, methacrylate, polypropylene, PDMS, Chitin, Resin, etc. Depending on the choice of surface, the chemistry for anchoring of the protein to the surface will be different. For example, one could use gold in the surface to anchor the cysteine residue, or one can take advantage of the reactivity of the sulfhydro group on the cysteine to create a covalent bond between the SH— and a group on the surface, such a maleimido group or an iodoacetamido group. The immobilization of the protein on a solid surface affords the possibility of using the GBP-based biosensors in a number of applications other than those involving a single-phase solution assay. Various methods for conjugation/immobilization of protein to insoluble substrates are well known in the art. (Rao, et al., Mikrochim. Acta 1998, 128, 127-143; Taylor, Protein Immobilization, 1991, Marcel Dekker.

The sensing systems described here, detection of the specific ligand is based on the protein conformational change associated with the binding of glucose or galactose to GBP. The present system could be used for the long-term continuous monitoring of glucose in vivo since no extraneous addition of substrate is needed, thereby avoiding possible perturbations to the system from the addition of reagents.

Because galactose can also bind to GBP and produce a conformational change, the use of labeled GBP for the detection of glucose would be limited to systems where galactose is not in similar amounts. Likewise, the sensing system for glucose using GBP can be used to detect galactose in systems where glucose is present in limited amounts.

The following examples are offered by way of illustration of the present invention, and not by way of limitation.

### EXAMPLES

### Example 1

Construction of Plasmids pSD5O2 and pSD5O3.

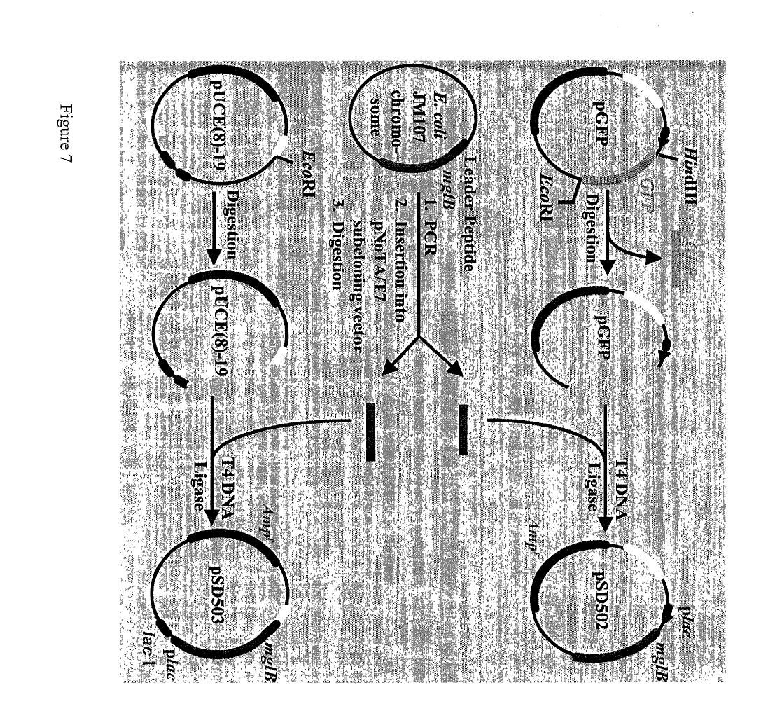

The first step was to construct plasmids containing the mglB gene, which codes for GBP, with and without the 23 amino acid leader sequence. This leader peptide is cleaved upon export of the protein from the cytoplasm to the periplasmic space. A schematic for the construction of these plasmids is shown in FIG. 7. Both forms of the gene were extracted from the chromosome of E. coli using PCR and primers designed on the basis of the gene sequence. The mglB gene without the leader sequence involved primers that incorporated EcoRI and HindIII restriction sites at each end of the gene. The primers for mglB with the leader sequence incorporated an EcoRI site at each end of the gene. Both PCR products (930 bp and 999 bp fragments of the gene without and with the leader sequence, respectively) were verified by gel electrophoresis. The fragments of interest were cut off the gel inserted into the pNoTA/T7 subcloning vector. A mini-prep was conducted on the resultant white colonies and the isolated plasmids digested with the respective restriction enzymes and the inserts isolated from a 1% low-melt agarose gel. The mglB gene without the leader sequence was inserted into pGFP vector, from which the gene for GBP had previously been extracted using EcoRI and HindIII restriction enzymes. The resultant plasmid, pSD502, now contained the gene that codes for cytoplasmic GBP. The mglB gene with the leader sequence was inserted into pUC(E) 8-19, which had been digested with EcoRI to enable ligation of the gene of interest. This plasmid, pSD503, consists of the gene that enables the expression of periplasmic GBP.

Isolation of the mglB gene. The mglB gene was extracted from the chromosome of JM107 strain of E. coli. The gene was obtained with and without the 23 amino acid leader sequence, aiding in the expression of both periplasmic and cytoplasmic protein, respectively. To extract mglB, the forward primer, mglBforE (30 mer) was used. It had an EcoRI restriction site prior to the primer sequence. The reverse primer, mglrevE (37 mer) also had an EcoRI cutting site. The reverse primer sequence was not complementary to the mglB gene. An EcoRI cutting site existed on the gene (residues 305, 306). Therefore, the primer incorporated a single base mismatch (G instead of A). However, the alteration of the base did not change the amino acid residue coded for. To isolate the mglB gene without the leader peptide, mglBforH (28 mer) was used. It had a HindIII restriction site prior to the primer sequence. The reverse primer was the same as in the above case. A 30 cycle PCR reaction was set up to amplify the two fragments of interest, with and without the leader peptide sequence. The hot start method was employed. The DNA polymerase, Pfu, was used and the sample product loaded on a 1% low melt agarose gel. The fragments of interest (999 bp and 930 bp of the mglB gene with and without the leader sequence, respectively) were cut off and purified using the QIAquick™ Gel Extraction Kit purchased from Qiagen (Chatsworth, Calif.).

Cloning mglB into pNoTA/T7. The PRIME PCR CLONER-™ Cloning System, 5 Prime→3 Prime (Boulder, Colo.), was used to efficiently clone the DNA fragments obtained by PCR into the plasmid shuttle vector, pNoTA/T7. The vector containing the insert was then transformed into JM109 cells. The cells were plated and incubated at 37° C. White colonies were indicative of the plasmid of interest while blue colonies were negative results. Positive colonies were grown in LB, the cells pelleted, and the plasmid isolated using the QIAprep™ Spin Plasmid Kit, Qiagen (Chatsworth, Calif.). The plasmid was then digested with EcoRI, for the gene with the leader peptide, and with EcoRI and HindHI, for the gene without the leader peptide. The two fragments of interest were isolated on a 1% low melt agarose gel and isolated using the QIAquick™ Gel Extraction Kit protocol. The samples were sequenced to verify the presence of the gene of interest.

Vector and Insert Preparation. All digestions were carried out at 37° C. for 1 h. A 1% low-melt agarose gel was run to verify the presence of the bands of interest. The pNoTA/T7 plasmid containing the mglB gene with the leader sequence was digested with EcoRI. Both the 2.7 Kbp and the 999 bp bands were evident. The shuttle vector containing the mglB gene without the leader sequence was digested with EcoRI and HindIII. Here the 2.7 Kbp and the 930 bp fragments were seen. The gene fragments were cut off and extracted from the gel. The plasmid pUC(E)8-19, was cut with EcoRI yielding a single band at ˜2.7 Kbp. The pGFP plasmid was cut with EcoRI and HindHIII to cut out the gene for GFP. Two bands were seen, one at ˜3 Kbp and the other at ˜600 bp. Both the vectors (2.7 and 3 Kbp fragments) were cut off the gel and purified using the QIAquick™ Gel Extraction protocol.

Ligation and Transformation. All ligations were carried out in a 14-16° C. water bath overnight using T4 DNA ligase. The mglB gene without the leader sequence was inserted into the pGFP plasmid, resulting in pSD502, the cytoplasmic protein. The mglB gene with the leader peptide was inserted into pUC(E)8-19, resulting in pSD503, the periplasmic protein. JM109 competent cells were prepared using the calcium chloride method. The plasmids containing the inserts were transformed into JM109 cells, which were then plated on LB plates containing ampicillin (100 ug/ml). After growing overnight at 37° C., the colonies of interest were picked and grown overnight in LB, and mini-preps using phenol-chloroform extraction were performed to isolate the plasmids.

### Example 2

Expression of mglB Gene.

Plasmids pSD502 and pSD503 were transformed into competent E. coli JM109 cells prepared by the calcium chloride method. Since the mglB with the leader sequence had EcoRI restriction sites on both of its ends, it was critical to determine the orientation of the gene in the plasmid. Digestion of the plasmid with restriction enzymes, PvuII and HindIII, resulted in 5 fragments (378, 119, 773, 90, and 2250 bp). This gel electrophoresis result was consistent with the predicted fragment sizes as determined by studying the plasmid map and the cutting sites. Finally, the identity of both genes was verified through sequencing.

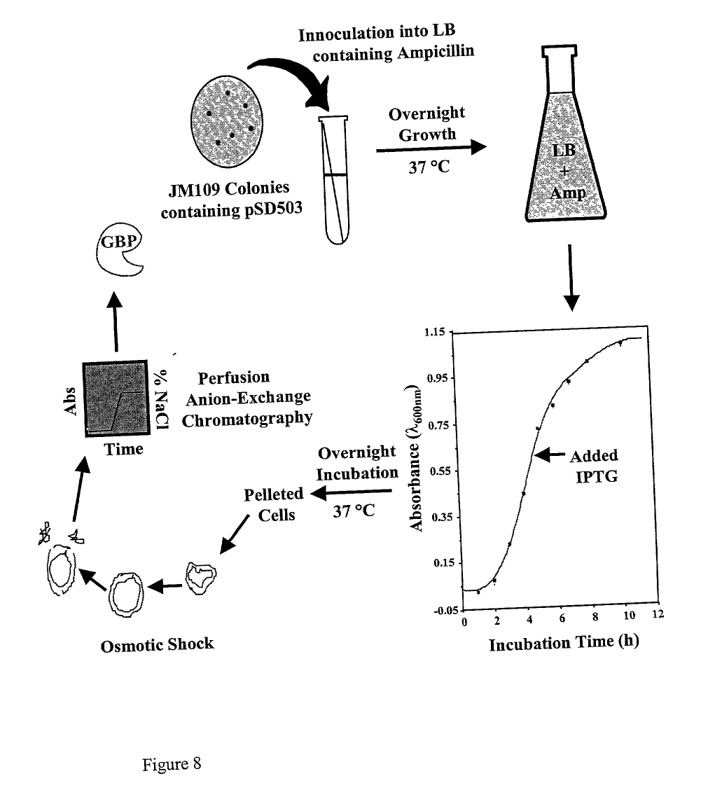

The expression of GBP is presented in FIG. 8. Isolated JM109 colonies containing pSD502 or pSD503 were inoculated into a LB starter culture containing ampicillin and grown overnight. The culture was then transferred into a larger volume of LB and allowed to incubate until an OD₆₀₀ of 0.6 was achieved. IPTG was then added to induce protein expression and the cells allowed to incubate overnight. The cells were then harvested and the protein released either by sonication (for cytoplasmic GBP) or osmotic shock (for periplasmic GBP). The protein was prepared for purification by centrifuging and then filtering the supernatant with a 0.2 um filter.

A single colony of E. coli strain JM109, containing the plasmid pSD502 or pSD5O3 was inoculated into 2 ml LB broth containing 100 μg/ml of the antibiotic ampicillin. Cells were grown overnight (17-18 h) at 37° C. in a shaker. This 2 ml culture was then diluted into 500 ml LB broth containing ampicillin and allowed to grow until an optical density of 0.6 was achieved. Protein expression was induced using 1 mm IPTG. The cultures were then allowed to grow overnight at 37° C. at 250 rpm in the shaker. The cells were pelleted by centrifuging at 5000 rpm, 4° C., for 15 min.

Periplasmic proteins were released using the osmotic shock procedure (Willsky et. al., J. Bacteriol 1976, 127, 595-609). The pellet was resuspended in 40 ml of 10 mM Tris-HCI/30 mM NaCl, pH 7.5. After the wash and centrifugation at 9500 rpm for 15 min at 27° C. the sedimented cells were resuspended in 40 ml of 33 mM Tris-HCl, pH 7.5 followed by centrifugation at 9500 rpm for 15 min at 27° C. The pellet was then resuspended vigorously in 40 ml Stage I buffer. The suspension was left at 37° C. for 10 min with very slow shaking. The cells were collected twice by centrifuging at 9500 rpm for 10 min at 27° C. The pellet was resuspended rapidly in 80 ml of chilled 0.5 mM MgCl₂ and subjected to vigorous shaking in an ice bath for 10-15 min. The purpose of Mg²⁺ in the low osmotic medium was to facilitate the complete release of the protein and to maintain its activity.

The shocked cells were removed by centrifugation at 9500 rpm for 10 min at 4° C. and the supernatant was collected and lyophilized. The result was a higher yield of protein. The lyophilized sample was dissolved in deionized water and dialysized three times against 10 mM Tris-HCl, pH 8.0. The resulting crude periplasmic extract was then centrifuged at 9000 rpm for 10 min at 4° C. to remove any cell membrane fragments and other solid particles. The supernatant was filtered with a 0.2 μm syringe filter and stored at 4° C.

### Example 3

Purification of Wild-Type GBP.

A procedure to purify GBP in a single chromatographic step was developed using perfusion chromatography technology. Unlike conventional chromatography particles, POROS™ particles have two types of pores. Large throughpores that transect the particle and short diffusive pores that branch off from the former. This creates a large surface area for the sample to interact with the particles. As a result, faster separations are possible with minor loss in resolution (Afeyan et al., J. Chrom. 1990, 519, 1-29; Regnier, F. E. Nature 1991, 350, 634-635).

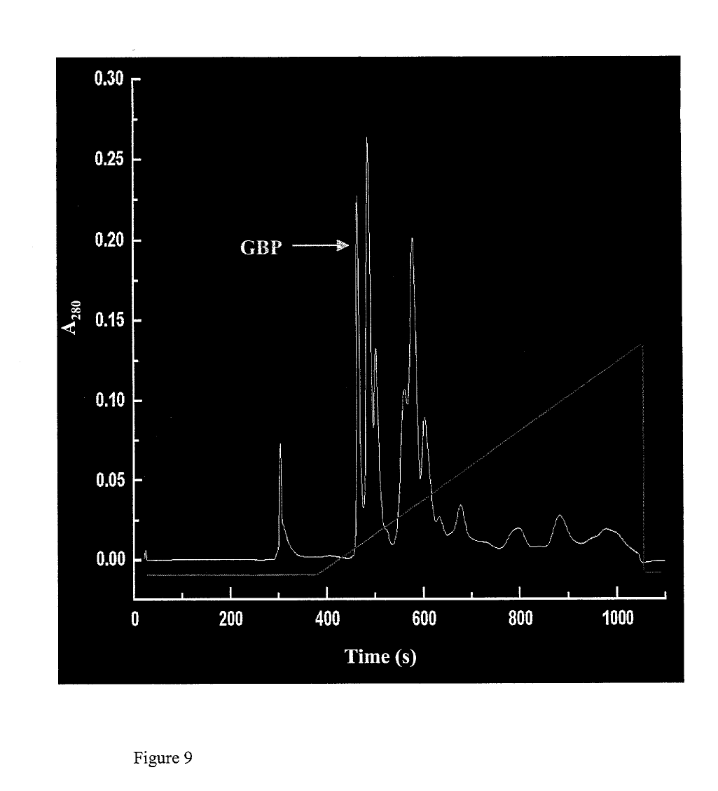

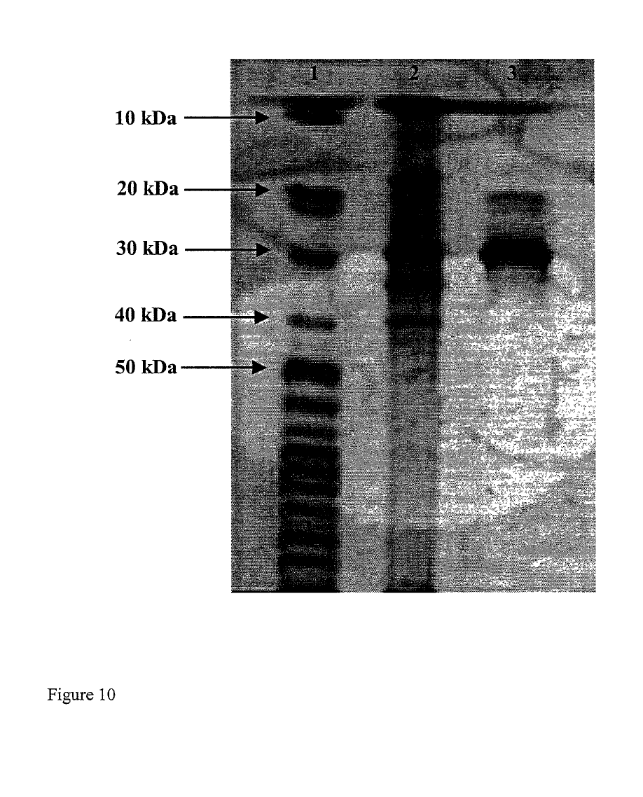

A strong anion exchange, high capacity, quartemized polyethyleneimine column was used. GBP was eluted using a gradient that started at 100% buffer A and terminated at 50% buffer A and 50% buffer B. The elution peak containing GBP was highly resolved (FIG. 9) and the purity of the fractions determined by SDS-PAGE. A 12.5% gel with silver staining indicated a single band at 33 kDa, which corresponds to the molecular weight of GBP. The purity of GBP was determined to be greater than 98% as seen in FIG. 10.

The BioCAD SPRINT™ Perfusion Chromatography System was used for protein purification. The column consisted of the functional group quarternized polyethyleneimine (HQ), a strong anion exchanger. Using a pH to 8.0 enabled the proteins to stick to the column. The high capacity column was equilibrated with buffer A (10 mM Tris-HCl, pH 8.0). The flow rate was set at 8 ml/min. 1 ml of the unpurified protein was injected onto the column. A 10 column volume wash with buffer A was followed by a salt gradient segment. Buffer B (10 mM Tris-HCl/1 M NaCI, pH 8.0) was used to elute the protein from the column. The protein was eluted using a gradient that started at 100% buffer A and terminated at 50% buffer A and 50% buffer B. Protein elution was monitored by UV absorbance at 280 nm. A 1 ml injection onto the column gave a peak with an absorbance of 0.14. Fractions of the purified proteins were collected and dialysized three times against 10 mM Tris-HCl, pH 8.0.

The purity of the GBP was determined by SDS-PAGE using 12.5% gels that were developed by the silver stain method. The amount of GBP present was ascertained using the Micro BCA Protein Assay Reagent Kit.

### Example 4

Labeling GBP with 2-dimethylamino-naphthalene-5-sulfonyl Chloride (D-22).

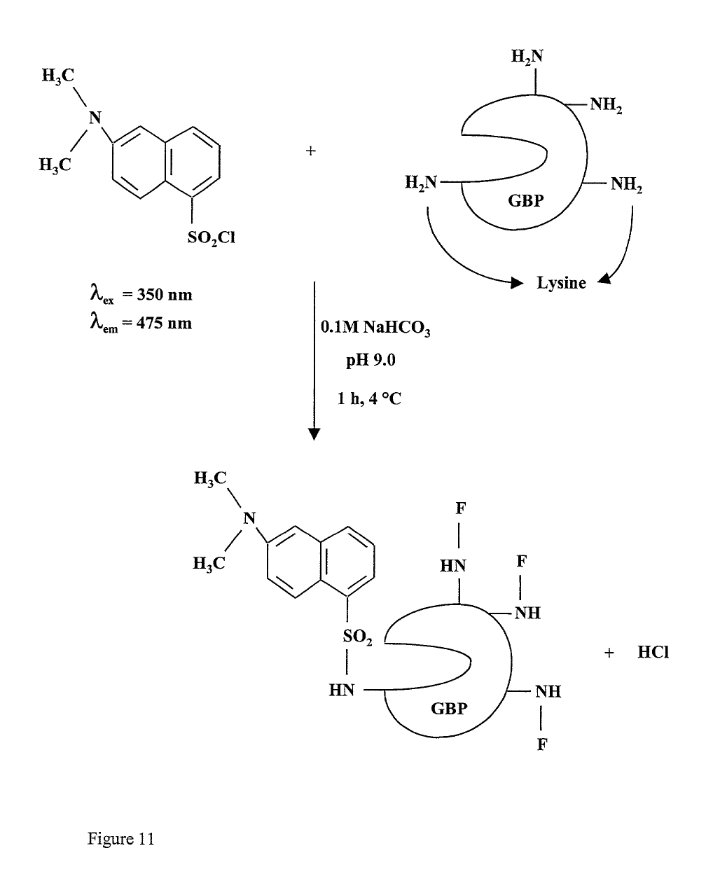

Proper folding of periplasmic proteins is attained during its transportation from the cytoplasm to the periplasmic space. Since correct folding is critical for the protein to carry out its function, the present inventors conducted steady-state fluorescence experiments with the periplasmic rather than the cytoplasmic GBP. Purified GBP was conjugated to the environment-sensitive fluorescent probe, D-22. The reaction was carried out in an ice-bath in order to control the otherwise rapid reaction. The fluorophore was added in small increments to ensure that all the lysine residues had an equal opportunity to react with the probe. FIG. 11 represents the reaction scheme for conjugation of 2-dimethylaminonaphthalene-5-sulfonyl chloride (D-22) to the lysine residues of GBP. Care was taken to make sure that the conjugate was always on ice since dansyl derivatives lose fluorescence intensity on standing at room temperature for long periods, even if protected from light.

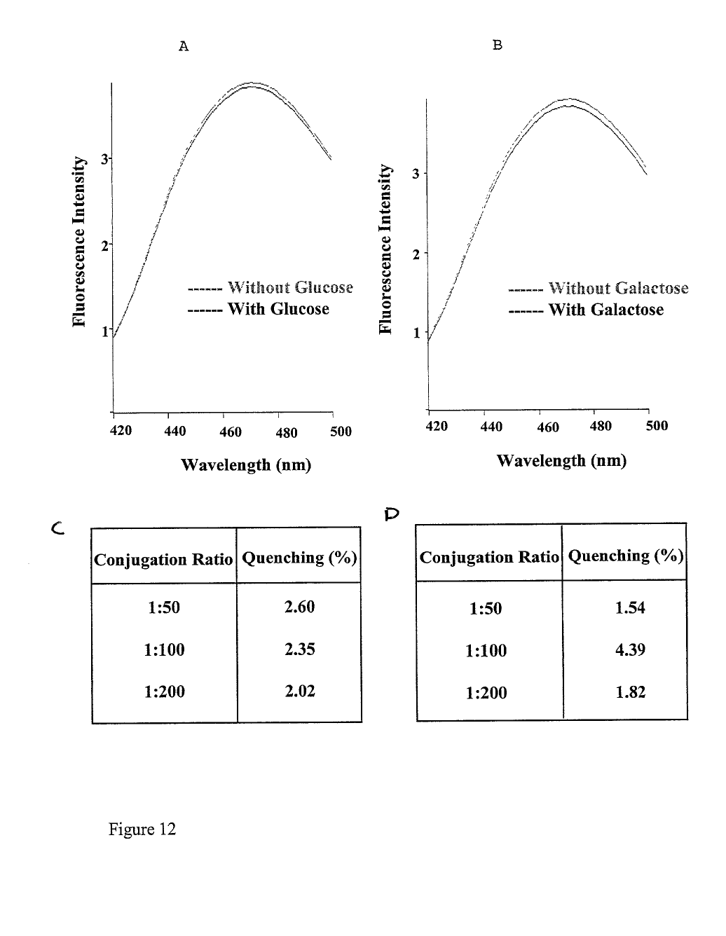

Once the three conjugates (each with different protein to fluorophore molar ratios) were prepared, studies were performed to characterize the fluorescence properties of the conjugate in the absence and presence of D-(+)-glucose (FIG. 12A) and D-(+)-galactose (FIG. 12B). Addition of 1.6×10⁻⁴ M of glucose or galactose to the dansylated GBP results in quenching of the fluorescence signal (FIG. 12C-glucose; FIG. 12D-galactose). None of the three conjugates showed any significant change in fluorescence intensity. This can be attributed to the fact that GBP has 22 lysine residues in its structure and labeling with D-22 results in multiple-site attachment of the probe to the protein molecule. It is hypothesized that some of these fluorophores are in locations where the ligand-induced hinge motion does not result in any change in the fluorescence intensity, leading to an increase in background fluorescence. If the fluorophore is attached only at sites where a change in fluorescence occurs upon ligand binding, then better detection limits should be achieved.

Labeling GBP with D-22. GBP was dialysed three times against 0.1 M NaHCO₃, pH 9.0. Three conjugation reactions with protein to fluorophore mole ratios of 1:50, 1:100, and 1:200 were carried out. Three microvials, each containing GBP, were placed in an ice bath. The fluorophore was added to the vials in small increments while the solution was being stirred. The mixture was allowed to react in the dark at 4° C. for 1 h. To eliminate the excess of unbound D-22, size exclusion chromatography was employed. A D-Salt Polyacrylamide 6000 desalting column was equilibrated with 10 mM Tris-HCl, pH 8.0. The conjugates were loaded onto the column. Elution of the labeled protein was conducted with the equilibration buffer and the conjugate finally dialysed three times against 10 mM Tris-HCl, pH 8.0.

Steady-state fluorescence studies of the labeled GBP. Fluorescence measurements were conducted with sample volumes of 1.5 mL and in quartz cuvettes. After the addition of 30 μL of D-(+)-galactose and D-(+)-glucose to the conjugate, the solution was allowed to incubate for 15 min at 4° C. while mixing at 400 rpm. The excitation and emission monochromator slit widths were set at 2 mm. The excitation wavelength was 350 nm and emission was detected at 475 nm.

### Example 5

Mutant GBP Plasmids pSD505, pSD506, and pSD504.

Three mutants of GBP were obtained by replacing the glycine, histidine, and methionine residues at positions 148, 152, and 182, respectively, with a cysteine using PCR site-directed mutagenesis. These sites were chosen by examining the x-ray crystal structure of GBP. The residues that might experience changes in their microenvironments when the ligand-induced conformational change occurs were identified. Since the wild-type protein lacks cysteines in its structure, introducing a cysteine mutation ensures single label attachment when a sulfhydro-specific fluorophore, such as MDCC, is used. The residues selected for mutation were not involved in galactose or glucose binding. They were chosen based on their proximity to the edge of the binding cleft, a region that experiences a significant change in local environment upon binding of the ligand.

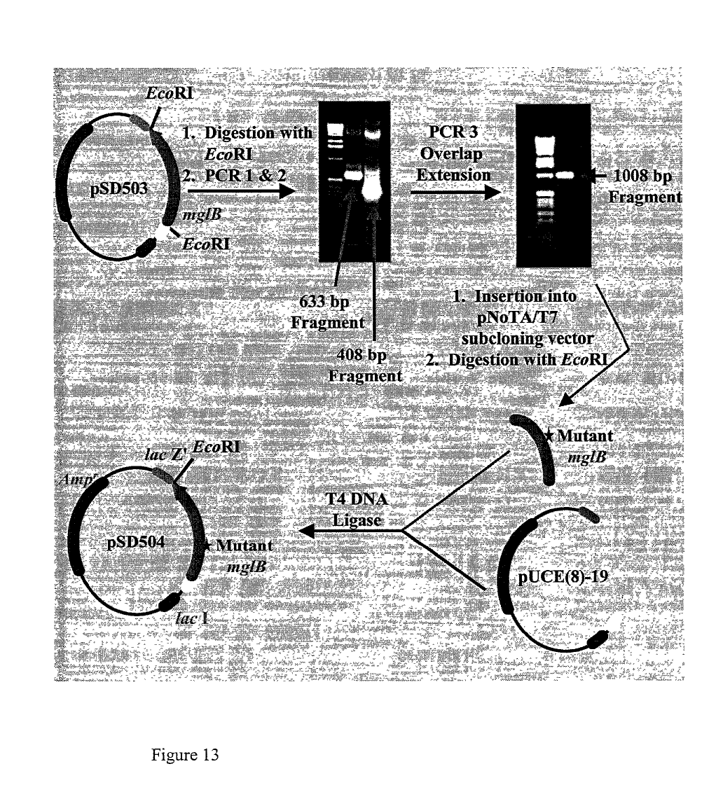

The three mutant mglB genes, each containing a single cysteine mutation at positions 148, 152, and 182, were constructed by site-directed mutagenesis. This was accomplished by PCR overlap extension using the wild-type mglB gene from pSD503 and primers designed to incorporate the mutation. The products from the two initial PCR reactions were isolated using a 1% low-melt agarose gel (FIG. 13) and purified using the QIquick™ Gel Extraction Protocol. These products were used to construct the overlap fragment containing the mutation. The mutant mglB genes were isolated on a 1% low-melt gel, as seen in FIG. 4.9, and extracted from the gel for purification. The genes containing the mutations were then inserted into the pNoTA/T7 subcloning system. The three purified mutant mglB genes were inserted into pUC(E)8-19 using T4 DNA ligase, thus creating pSD505, pSD506, and pSD504.

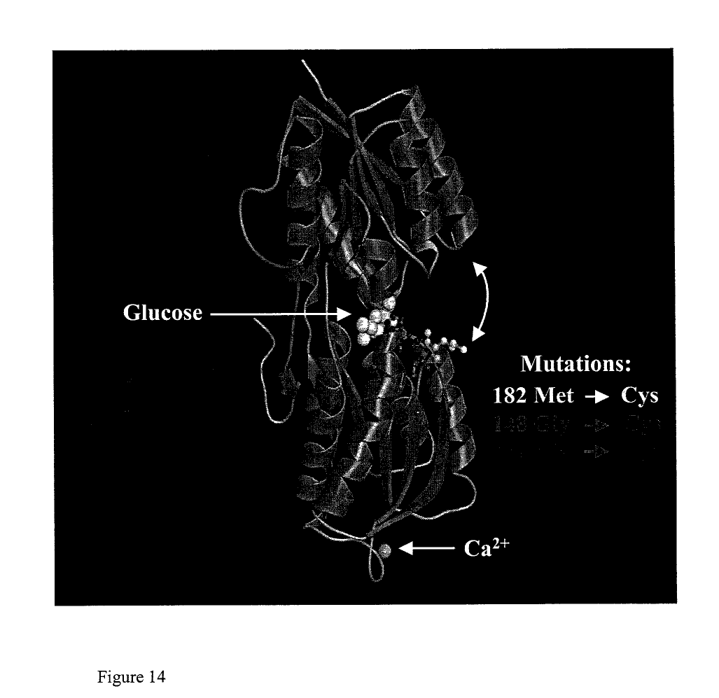

The site-specific mutations (FIG. 14) enable the attachment of a single fluorophore to the protein and should eliminate background fluorescence associated with multiple site labeling. These sites were labeled with the fluorophores, MDCC, acrylodan, 1,5-IAEDANS, or IANBD ester. In each case, the unconjugated fluorophores were separated from the labeled protein by running the reaction mixture through a size-exclusion column. Steady-state fluorescence studies indicated that the signal intensity of the various probes was quenched in the presence of glucose and galactose (Table 4.1).

TABLE 4.1
Response of the three GBP mutants labeled with different
fluorophores upon binding glucose and galactose as % quenching.

| | | MDCC | Acrylodan | IANBD | IAEDANS |
|---|---|---|---|---|---|
| M182C | Glucose | 12 | 7 | — | — |
| | Galactose | 6 | 5 | — | — |
| H152C | Glucose | 30 | — | 5 | — |
| | Galactose | 19 | — | 4 | — |
| G148C | Glucose | 18 | — | — | 7 |
| | Galactose | 16 | — | — | 7 |

The (-) representsno change in fluorescence intensity.

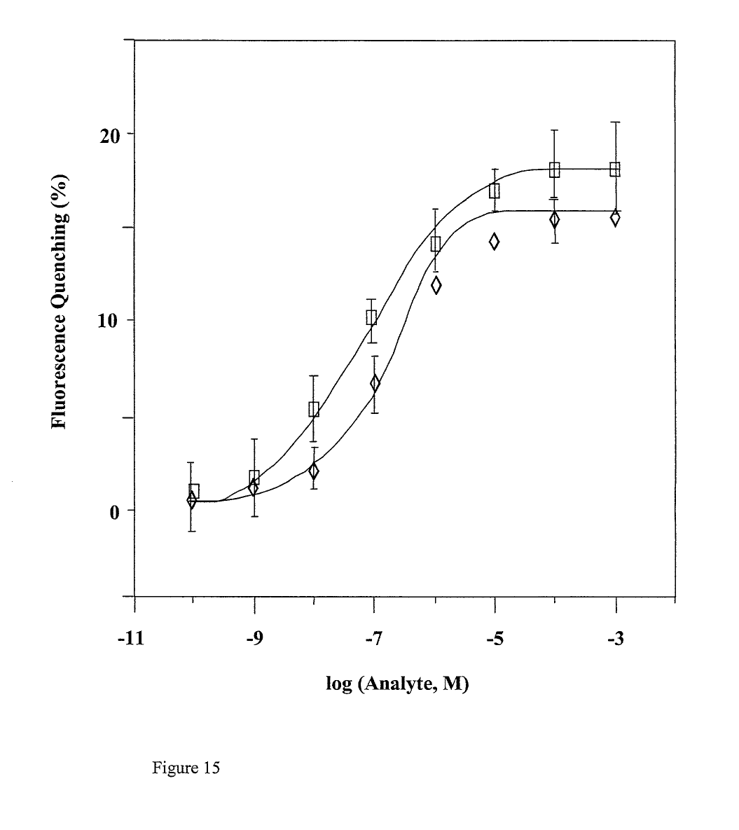

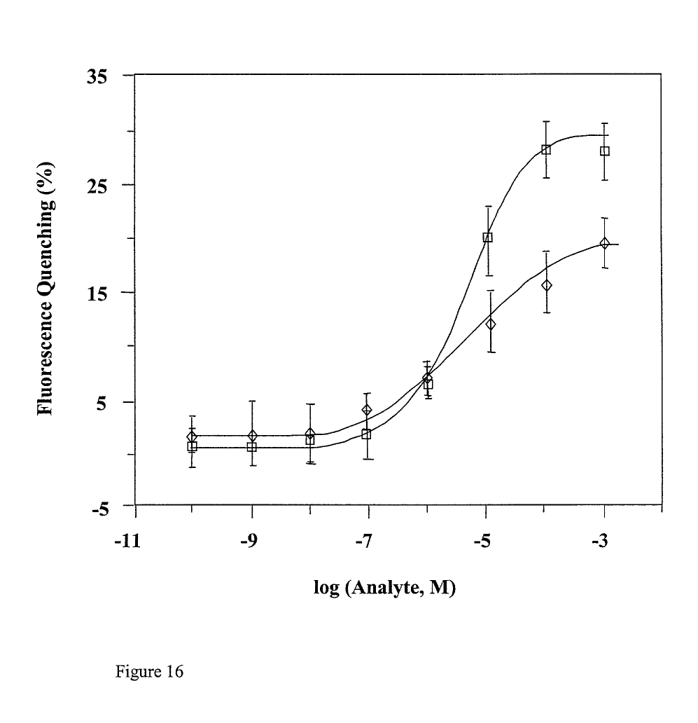

This proves that the ligand-induced conformational change presents the fluorophore to a more hydrophilic environment and perhaps exposed to solvent molecules. As expected, the systems responded similarly to both glucose and galactose. The calibration plot for the sugars using the GBP labeled with MDCC at position 148, shows a maximum fluorescence quenching of 18% and 16% for glucose and galactose, respectively (FIG. 15). The detection limit for glucose was 5×10⁻⁸ M (S/N=3). The largest change in signal, however, was seen in the case of the cysteine at position 152 labeled with MDCC. FIG. 16 shows a 30% and 19% quenching of the fluorescence intensity of MDCC in the presence of glucose and galactose, respectively. The detection limit for glucose was 1×10⁻⁶ M (S/N=3). In both cases, the response time of the system to the sugar was 15 min. Although this length of time was required for maximum change in fluorescence, the analysis time can be reduced at the expense of signal intensity and/or detection limit. The storage life of the labeled protein in solution at 4° C. was determined to be at least two months. This not only makes the system easier to market but also enables long-term storage without compromising reproducibility of the data.

We hypothesize that MDCC is the best fluorophore for use as a label in the sensing system for the sugars is because of its structure. Most commercially available fluorescent maleimide probes have the maleimido group attached directly to the aromatic ring. In MDCC an aliphatic spacer arm between the maleimide and fluorophore, acts as a flexible link positioning the probe in a favorable position on the protein (Corrie, J. E. T. J. Chem. Soc. Perkin Trans. 1990, 1, 2151-2152). The other three probes are rigid structures and may have difficulty being positioned in the hydrophobic folds of the protein.

Construction of mutant GBP plasmids. The three plasmids for the expression of mutant GBP were constructed using site-directed mutagenesis. The glycine (position 148), histidine (position 152), and methionine (position 182) were each replaced with a single cysteine. The alterations were performed by overlap extension PCR using the mglB gene (from pSD503) as the template. The products of PCR reactions 1 and 2 were obtained by primers designed to incorporate the mutation and used with the forward and reverse primers designed for extraction of the wild-type mglB gene. These products were loaded on a 1% low-melt agarose gel and purified using the QIAquick Gel Extraction protocol. They were then used in the construction of the overlap fragment, the mutant mglB containing a cysteine at either of the three positions. The overlap fragment was verified on a 1% low-melt agarose gel and purified in with the QLAquick Gel Extraction protocol. The mutant mglB fragment was further purified by insertion into the pNoTA/T7 subcloning vector, using the same procedure as previously described. The gene was digested from the subcloning vector using the restriction enzyme, EcoRI. The pUC(E)8-19 plasmid was prepared by digestion with EcoRI. The mutant mglB gene was inserted into the plasmid using T4 DNA ligase. The resulting plasmids, pSD505 (Glyl48Cys), pSD506 (His152Cys), and pSD504 (Met182Cys) were transformed into competent E. coli JM109 cells and grown on LB plates containing ampicillin. The protocol for the expression and purification of the mutant proteins was the same as that developed for the wild-type protein.

Labeling the mutants of GBP with fluorescent probes. The mutant protein was first dialysed three times against either 10 mM Tris-HCl, 1 mM DTT, 0.2 mM CaCl₂, pH 8.0 (for reaction with MDCC) or 10 mM HEPES, 1 mM DTT, 0.2 mM CaCl₂, pH 7.4 (for reaction with acrylodan, 1,5-IAEDANS, or IANBD ester). DTT was present in the buffers to reduce any disulfide linkages that may have formed between two protein molecules. The protein was then dialysed three times against the respective buffers (with no DTT) to remove excess of the reducing agent. For the conjugation reaction, a molar ratio of fluorophore to protein of 5:1 was employed. The fluorophores were added slowly to the mutants of GBP (μM concentrations) so as to allow the cysteine residue on all the protein molecules to have an equal opportunity to react with the fluorophore. The solution was stirred constantly in a glass reaction vessel and in the dark for 4 h at 4° C. (with MDCC and acrylodan) or for 2-3 h at RT (with 1,5-IAEDANS or IANBD ester). The conjugated protein was separated from the free fluorophore by running the sample through a Sephadex G-25 column and eluting with the respective buffer.

Steady-state fluorescence studies of the fluorophore-labeled mutant proteins. The excitation and emission monochromator slit widths were both set at 2 mm. The excitation and emission wavelengths for MDCC, acrylodan, 1,5-IAEDANS, and IANBD ester were 425 nm and 475 nm, 382 nm and 509 nm, 336 nm and 490 nm, and 472 nm and 536 nm, respectively. All data were obtained at RT using quartz cuvettes with sample volumes of 1.5-2 mL. The concentrations of labeled proteins used in the steady-state fluorescence studies were in the 1×10⁻⁷ M range. The conjugates were incubated with various concentrations of glucose and galactose for 15-20 min at RT on a shaker at 400 rpm. The samples were analyzed in the spectrofluorometer under the aforementioned parameters. Calibration plots were obtained by relating the average fluorescence change with the concentration of glucose or galactose in the sample.

### Example 6

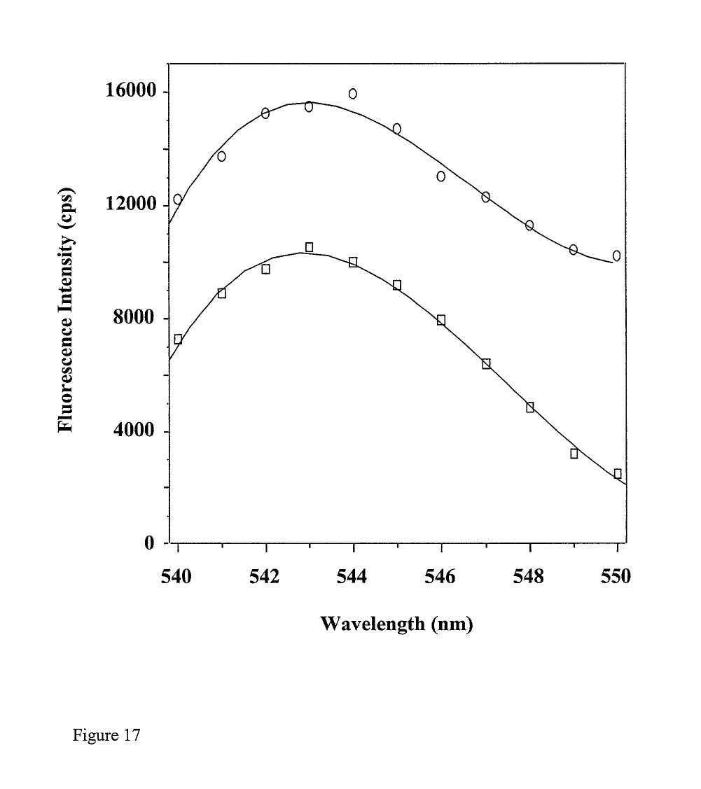

To test the feasibility of using the wild-type GBP in a sensing scheme for glucose, the calcium ion was chelated with EDTA and extracted from its binding site in GBP. The calcium was then replaced with terbium through several dialysis steps. The fluorescent terbium could then be employed to report the conformational changes occurring upon ligand binding. All experiments were carried out with the protein present in 10 mM Tris-HCl, pH 7.4. FIG. 17 clearly indicates that there is a significant increase in the fluorescence intensity of terbium upon the addition of glucose. This increase is a result of local environmental changes that occur around the metal when the protein undergoes the conformational change caused by the binding event. It is hypothesized that this change exposes the terbium reporter to a more hydrophobic environment and perhaps shielded from surrounding solvent molecules. As a result, an increase in fluorescence intensity is observed upon ligand binding. This change can then be used as the basis for the development of a sensing system for glucose.

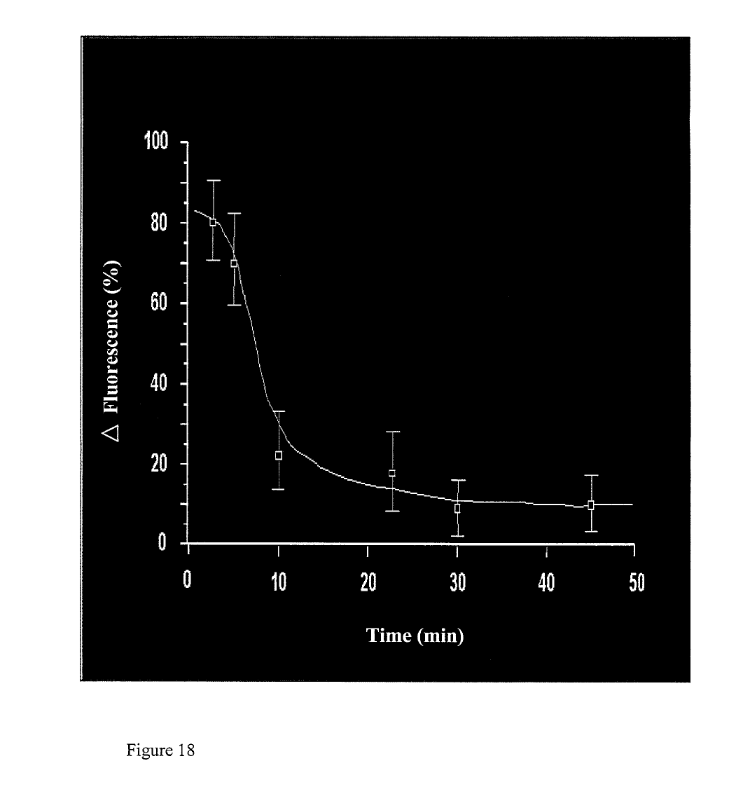

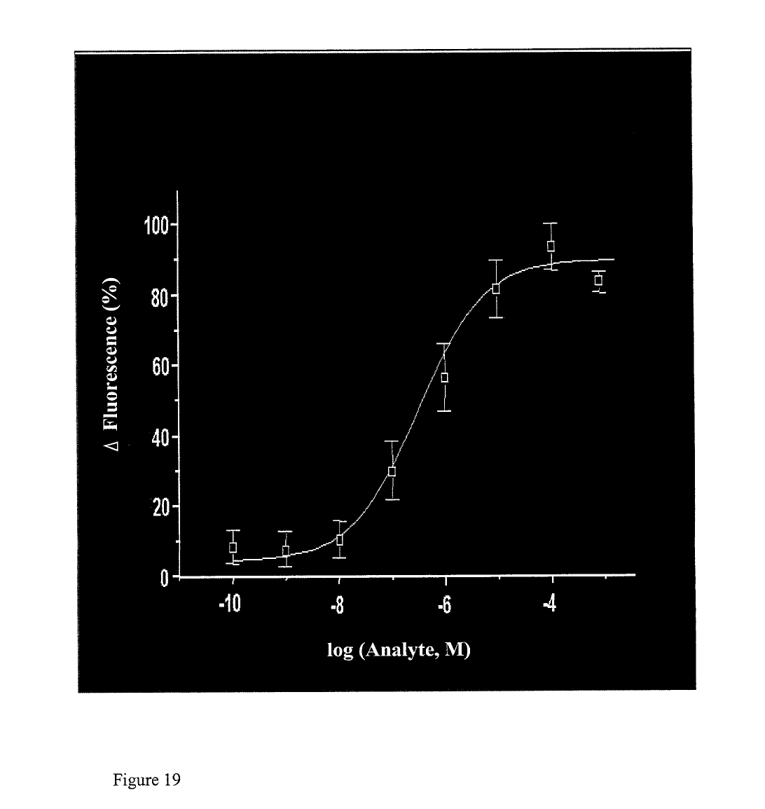

To optimize the system, a calibration plot was constructed to determine the time necessary to see maximum change in fluorescence intensity. The plot in FIG. 18 indicates that an incubation time of 2 min between GBP and the ligand is sufficient to obtain 80% enhancement of the fluorescence signal. After that the change is significantly lower and can be attributed to energy transfer between the terbium ion and neighboring amino acid residues that are in closer proximity after the binding event. A calibration curve was constructed by incubating the protein for 2 min with various concentrations of glucose. FIG. 19 indicates the increase in fluorescence intensity with increasing concentrations of the ligand. The detection limit for this system was determined to be 1×10⁻⁷ M (S/N=3).

Since GBP binds to both epimers, glucose and galactose, Table 4.2 depicts a similar response of the system towards glucose and galactose when employing GBP complexed with Tb³⁺.

TABLE 4.2
Percent signal of lanthanide fluorescence on ligand
binding by wild-type GBP.

| | Tb³⁺ | Eu³⁺ |
|---|---|---|
| Glucose | 85 | 13 |
| Galactose | 92 | 19 |

However, the system containing europium as a reporter did not show as much of a response compared to the terbium-based reporter system. This can be explained by the fact that europium is much larger in size than calcium and that it may have a difficulty remaining in the binding pocket. The fluorescence of europium is quenched easily by solvent molecules and this could also be a contributing factor to the lack of enhancement in signal in the presence of glucose. This work is good indication that terbium-labeled GBP can be used as a reagentless sensing system for detection of sub-μM concentrations of glucose.

Introducing terbium and europium in the metal binding site of GBP. GBP was dialysed three times against 10 mM HEPES, 20 mM EDTA, 100 mM KCl, pH 7.0 to get rid of the bound calcium. The calcium was replaced with terbium (or europium) by dialysis against 10 mM HEPES, 1 mM TbCl3 (or EuCl3), 100 mM KCl, pH 7.0.

Steady state fluorescence studies of the labeled GBP. Samples with a volume of 1.5 ml were measured in a quartz cuvette. After the addition of various concentrations of D-(+)galactose and D-(+)-glucose to the conjugate, the solution was allowed to incubate for 2 min at room temperature while mixing at 400 rpm. The excitation and emission monochromator slit widths were set at 2 mm. The excitation wavelength was 272 nm and emission was detected at 543 nm.

### Example 7

Bacterial Strains and Plasmids, Materials and Apparati

Bacterial Strains and Plasmids. E. coli strain JM107 was used for extraction of the mglB gene with and without the leader peptide. E. coli strain JM109 containing the plasmids pSD5O2 and pSD503, and carrying the mglB gene with and without the leader peptide, respectively, was used for expression of periplasmic and cytoplasmic GBP. Plasmids pSD502 and pSD503 were constructed by PCR-site directed mutagenesis using primers from Operon Technologies (Alameda, Calif.). The primers used for the isolation of the cytoplasmic protein gene were mglB forH (5′-TCT AAG CTT GGC TGA TAC TCG GAT TGG T-3′) and mglB revE (5′-AGA GAA TTC TTA TTT CTT GCT GAG TTC AGC CAG GTT G-3′) while those used for the periplasmic protein gene were mglB forE (5′-TCT GAA TTC ATG AAT AAG AAG GTG TTA ACC-3′) and mglB revE. The mglB gene with and without the leader sequence was introduced into pUC(E)8-19 and pGFP (after the gene for GFP was removed) resulting in the construction of plasmids pSD5O3 and pSD502, respectively.

The mglB gene containing the single cysteine mutation at position 148, 152, or 182 was also introduced into pUC(E)8-19 creating pSD505, pSD506, or pSD504. The forward and reverse primers for the mutation at position 148 (Gly to Cys) were FOR148MGBP (5′-GTA CTG CTG AAA TGT GAA CCG GGC CAT CCG-3′) and REV148MGBP (5′-GCC CGG TTC ACA TTT CAG CAG TAC GAA CTG-3′), respectively. The forward and reverse primers for the mutation at position 152 (His to Cys) were FOR152MGBP (5′-GGT GAA CCG GGC TGT CCG GAT GCA GAA-3′) and REV152MGBP (5′-ATC CGG ACA GCC CGG TTC ACC TTT CAG-3′), respectively. Finally, the two primers for the mutation at position 182 (Met to Cys) were forGBPmut (5′-TTA GAT ACC GCA TGC TGG GAC ACC GCT CAG-3′) and revGBPmut (5′-GGT GTC CCA GCA TGC GGT ATC TAA CTG TAA-3′).

Materials. Luria-Bertani (LB) medium, restriction enzymes, DNA polymerases, and T4 DNA ligase were all purchased from GibcoBRL (Gaithersburg, Md.). Tris buffer ([tris(hydroxymethyl)aminomethane]) was obtained from VWR Scientific (S. Plainfield, N.J.). The antibiotic, ampicillin, was purchased from Sigma (St. Louis, Mo.). [Ethylenedinitrilo]-tetraacetic acid (EDTA) was bought from Mallinckrodt (Paris, Ky.). All organic and inorganic salts were purchased from either Fisher Scientific (Fair Lawn, N.J.), VWR Scientific (S. Plainfield, N.J.) or Sigma (St. Louis, Mo.).

Both wild-type and the mutant GBP expressions were induced with IPTO purchased from GibcoBRL (Gaithersburg, Md.). Stage I buffer was 33 mM Tris-HCl, pH 7.5 containing 40% sucrose and 0.1 mM EDTA and was used in the osmotic shock procedure to release the protein from the periplasm.

The bicinchoninic acid (B CA) protein micro assay reagent kit from Pierce (Rockford, Ill.) was used to determine concentration of purified GBP. The fluorophore, 2-dimethylamino-naphthalene-5-sulfonyl chloride (D-22), which was used to label wild-type GBP, was obtained from Molecular Probes (Eugene, Oreg.). The sulfhydro-specific probes, 6-acryloyl-2-dimethylaminonaphthalene (acrylodan), 5-((((2-iodoacetyl)amino)ethyl)amino)naphthalene-1-sulfonic acid (1,5-IAEDANS), and N-((2-iodoacetoxy)ethyl)-N-methyl)amino-7-nitrobenz-2-oxa-1,3-diazole (IANBD ester) were also purchased from Molecular Probes. N-[2-(1-maleimidyl)ethyl]-7-(diethylamino)coumarin-3-carboxamide (MDCC) was synthesized in our laboratory following the published method (Corrie, J. E. T. J. Chem. Soc. Perkin Trans. 1990, 1, 2151-2152; Corrie, J. E. T. J. Chem. Soc. Perkin Trans. 1994, 1, 2975-2982). The conjugated protein was separated from unbound fluorophore by running the reaction mixture through either a D-salt polyacrylamide 6000 desalting column from Pierce (Rockford, Ill.) or a Sephadex™ G-25 size-exclusion column from Sigma Chemical Co. (St. Louis, Mo.).

Apparatus. DNA amplification was conducted with a GeneAmp™ PCR System 2400 by Perkin Elmer (Norwalk, Conn.). Bacterial colonies were grown on agar plates at 37° C. in a Fisher Scientific Incubator (Fairlawn, N.J.). Cell cultures were grown in an Orbital Shaker, Forma Scientific (Marrietta, Ohio) and pelleted using a Beckman 32-Nil Centrifuge (Palo Alto, Calif.). Cytoplasmic GBP was released using a 550 Sonic Dismembrator™ from Fisher Scientific (Fairlawn, N.J.). Unpurified protein fractions were filtered with a 0.2 um syringe filter from Nalgene (Rochester, N.Y.).

The BioCAD SPRINT™ Perfusion Chromatography System by PerSeptive Biosystems (Cambridge, Mass.) was used for protein purification. Periplasmic shockate was lyophilized using the VirTis Bench Top 3 Freeze Dryer (Gardiner, N.Y.). Proteins were dialysed against the correct buffer using a 12-14,000 dalton molecular weight cutoff SPECTRA/POR™ molecular porous membrane by Spectrum Medical Industries (Los Angeles, Calif.). Protein purity was verified by SDS-PAGE using a PhastSystem™ from Pharmacia Biotech (Uppsala, Sweden). Protein absorbances were determined with a diode array spectrophotometer (model 8453) from Hewlett Packard (Palo Alto, Calif.). Fluorescence studies were performed on a Fluorolog-2 fluorometer, Spex Industries Inc. (Edison, N.J.), equipped with a 450-Watt Xenon arc lamp.

All of the references cited herein are incorporated by reference in their entirely.

### REFERENCES

1. Afeyan, N. B.; Gordon, N. F.; Mazsaroff, I.; Varady, L.; Fulton, S. P.; Yang, Y. B.; Regnier, F. E. J. Chrom. 1990, 519, 1-29.

2. Afeyan, N. B.; Fulton, S. P.; Gordon, N. F.; Mazsaroff, I.; Varady, L.; Regnier, F. E. Bio/Technology 1990, 8,203-206.

3. Ames, G. F. L. Annu. Rev. Biochem. 1986, 55, 397425.

4. Bard, A. J., Faulkner, L. R., Electrochemical Methods: Fundamentals and Applications, Wiley, New York, 2000

5. Bruno, J.; Horrocks, W. DeW.; Zauhar, R. J. Biochem. 1992, 31, 7016-7026.

6. Corrie, J. E. T. J. Chem. Soc. Perkin Trans. 1990, 1, 2151-2152.

7. Corrie, J. E. T. J. Chem. Soc. Perkin Trans. 1994, 1, 2975-2982.

8. Deal et al., J. Med Chem. 1996, 39, 3096-3106.

9. Deo, S. K., Daunert, S., Anal. Biochem., 2001, 289, 52-59.

10. Dickson, E. F., A. Pollak, and E. P. Diamandis, Pharmacol. Ther., 1995. 66, 207-235

11. Dickson, E. F., A. Pollak, and E. P. Diamandis, J. Photochem. Photobiol. B, 1995. 27, 3-19.

12. Furlong, C. E. In Escherichia coli and Salmonella typhimurium: Cellular and Molecular Biology, Neidhardt, F. C., ed.; American Society of Microbiology: Washington, D.C., 1987, pp. 768-796.

13. Gleria, K. D., Hill, H. A. O., Wong, L. L., N-(2-Ferrocene-ethyl)maleimide: A New Electroactive Sulfhydryl-Specific Reagent For Cysteine-Containing Peptides and Proteins, FEBS Lett., 1996, 390: p. 142-44.

14. Hemmila, I. A. and H. J. Mikola, Acta. Radiol. Suppl., 1990.374, 53-55

15. Hemmila, I., Scand. J. Clin. Invest., 1998, 48, 389-399.

16. Heyduk and Heyduk Anal. Biochem. 1997, 248, 216-227.

17. Horrocks, W. DeW.; Sudnick, D. R. Acc. Chem. Res. 1981, 14, 384-392.

18. Karsilayan et al. Bioconjugate Chemistry 1997 8, 71-75.

19. Kissinger, P. T., Heineman, W. R., Laboratory Techniques in Electroanalytical Chemistry, Marcel Dekker, 1996).

20. Mahoney, W. C.; Hogg, R. W.; Hermodson, M. A. J. Biol. Chem. 1981, 256, 4350-4356.

21. Markela, E., T. H. Stahlberg, and I. Hemmila, J. Immunol. Methods, 1993. 161, 1-6 Martin, R. B.; Richardson, F. S. Q. Rev. Biophys. 1979, 12, 181-209.

22. Miller, D. M.; Olson, 3.5.; Pflugrath, 3. W.; Quiocho, F. A. J. Biol. Chem. 1983, 258, 13665-13672.

23. Niemi, P., et al., Invest. Radiol., 1991. 26, 820-824

24. Rao, S. V., Anderson, K. W., and Bachas, L. G., “Oriented Immobilization of Proteins”, Mikrochim. Acta, 128, 127-143 (1998)

25. Regnier, F. E. Nature 1991, 350, 634-635.

26. Sack, 3. 5.; Saper, M. A.; Quiocho, F. A. J. Mol. Biol. 1989, 206, 171-191.

27. Scholle, A.; Vreeman, 3.; Blank, V.; Nold, A.; Boos, W.; Manson, M. Mol. Gen. Genet. 1987, 208, 247-253.

28. Selvin, P. R. Methods in Enzymol. 1995, 246, 300-334.

29. Seveus, L., et al., Cytometry, 1992. 13, 329-338.

30. Skoog, D. A., Holler, F. J., and Nieman, T. A., Principles of Instrumental Analysis, 5th Edition, Saunders College Publishing, Philadelphia, 1998.

31. Suonpaa, M., et al., J. Immunol. Methods, 1992.149, 247-253.

32. Taylor, R. F., Protein Immobilization, Marcel Dekker, 1991.

33. Trammell, S. A., Goldston, H. M., Tran, J. P. T., Tender, L. M., Synthesis and Characterization of a Ruthenium (II)-BAsed Redox Conjugate for Reagentless Biosensing, Bioconjugate Chem., 2001, 12: p. 643-47.

34. Vereb et al. Biophys. J. 1998, 74, 2210-2222.

35. Vyas, N. K.; Vyas, M. N.; Quiocho, F. A. Nature 1987, 327, 635-638.

36. Vyas, N. K.; Vyas, M. N.; Quiocho, F. A. Science 1988, 242, 1290-1295.

37. Vyas, N. K.; Vyas, M. N.; Quiocho, F. A. J. Biol. Chem. 1991, 266, 5226-5237.

38. Williams, E. B.; Krishnaswamy, S.; Mann, K. G. J. Biol. Chem. 1989, 264, 7536-7545.

39. Wang, J., Analytical Electrochemistry; Wiley, New York, 2000

40. Willsky, G. R.; Malamy, M. H. J. Bacteriol. 1976, 127, 595-609.

41. Wilson, D. B.; Smith, 3. B. In Bacterial Transport, Rosen, B. P., ed.; Marcel Dekker: New York, 1978, pp. 495-557.

42. Wilson, G. S.; Hu, Y. Chem. Rev. 2000.

We claim:

1. A method for detecting a carbohydrate in a sample comprising adding a carbohydrate binding protein to said sample, wherein said binding protein changes conformation when bound with said carbohydrate, wherein said binding protein is labeled with an assayable ion that generates a signal upon a conformational change in the proteins; and
detecting the signal from said assayable ion.

2. The method according to claim 1, wherein said carbohydrate is glucose or galactose.

3. The method according to claim 1, wherein said binding protein is galactose/glucose binding protein, a lectin, a carbohydrate binding domain of a lectin, or a carbohydrate receptor.

4. The method according to claim 1, wherein said assayable ion replaces calcium ion in said protein.

5. The method according to claim 4, wherein said assayable ion is a lanthanide series ion.

6. The method according to claim 5, wherein said lanthanide series ion is a terbium ion.

7. The method according to claim 1, wherein said assayable ion generates a fluorescent signal.

8. A composition comprising (i) galactose/glucose binding protein bound to a substrate through a cysteine and (ii) a reporter moiety.

9. The composition according to claim 8, wherein said reporter moiety is covalently attached to said galactose/glucose binding protein.

10. The composition according to claim 8, wherein said reporter moiety is complexed by said galactose/glucose binding protein.

11. The composition according to claim 10, wherein the reporter moiety is a lanthanide series ion.

12. The composition according to claim 10, wherein said lanthanide series ion is a terbium ion or a europium ion.

13. A kit for detecting or quantitating the presence of glucose or galactose in a sample, comprising:
a galactose/glucose binding protein and a lanthanide series ion, wherein said galactose/glucose binding protein is labeled with said lanthanide series ion; and
written material describing how to determine the presence of glucose or galactose in said sample using said labeled galactose/glucose binding protein.

14. A kit for detecting or quantitating the presence of glucose or galactose in a sample, comprising:
a galactose/glucose binding protein, a lanthanide series ion, a reagent for removal of calcium from said galactose/glucose binding protein, a reagent for inserting a lanthanide series ion into the calcium binding site; and written material describing how to determine the presence of glucose or galactose in said sample using said galactose/glucose binding protein labeled with said lanthanide series ion.

15. A method for determining the concentration of glucose or galactose in a sample comprising,
adding galactose/glucose binding protein labeled with a lanthanide series ion to said sample,
monitoring the fluorescence of said lanthanide series ion to determine if said galactose/glucose binding protein undergoes a conformation change,
correlating the amount of change in fluorescence of said lanthanide series ion with the amount of said glucose or galactose in said sample.

16. The method according to claim 15, wherein the change in fluorescence is a change in fluorescence intensity.

17. The method according to claim 15, wherein the change in fluorescence is a change in fluorescence lifetime.

18. A sensor for determining the presence or concentration of a carbohydrate in sample comprising:
a galactose/glucose binding protein labeled with a reporter moiety, wherein said galactose/glucose binding protein is bound to a solid support;
a means for detecting a signal of said reporter moiety generated upon binding of said carbohydrate to the labeled galactose/glucose binding protein; and
a means for correlating the signal of the reporter with the amount of carbohydrate present in said sample.

19. The sensor of claim 14, wherein said galactose/glucose binding protein (GBP) is modified to include one or more cysteine residues.

20. The sensor of claim 14, wherein said galactose/glucose binding protein (GBP) is bound to said solid support by a cysteine residue.

21. A method for determining the concentration of glucose or galactose in a sample comprising,
adding to the sample a galactose/glucose binding protein and an organic compound that binds non-covalently to the galactose/glucose binding protein and generates a signal upon a conformational change in the galactose/glucose binding protein;
measuring the signal of said organic compound correlating the amount of change in fluorescence of said organic compound with the amount of said glucose or galactose in said sample.

22. The method of claim 21 where the organic molecule is anilinonaphthalenesulfonate.

23. A method for determining the concentration of glucose or galactose in a sample comprising,
adding to the sample a galactose/glucose binding protein labeled with an electrochemical molecule that generates a signal upon a conformational change in the galactose/glucose binding protein;
monitoring the signal of said electrochemical molecule to determine if said galactose/glucose binding protein undergoes a conformation change;
correlating the amount of change in signal of said electrochemical molecule with the amount of said glucose or galactose in said sample. signal-generating molecule in its structure.

24. A method for determining the concentration of glucose or galactose in a sample comprising,
adding to the sample a signaling fusion protein comprising a galactose/glucose binding protein fused to a signal generating molecule that generates a fluorescence signal upon a conformational change in the galactose/glucose binding protein;
measuring the fluorescence signal of said signaling fusion protein;
correlating the amount of change in the fluorescence signal with the amount of said glucose or galactose in said sample.

25. The method according to claim 24, where the signal generating molecule is Enhanced Green Fluorescence Protein.
